(19) United States
(12) Patent Application Publication
(10) Pub. No.: US 2022/0411484 A1
(43) Pub. Date: Dec. 29, 2022

(54) METHODS FOR TREATING VIRAL INFECTIONS

(71) Applicant: The Regents of The University of California, Oakland, CA (US)

(72) Inventor: Dennis J. Hartigan-O'Connor, Davis, CA (US)

(73) Assignee: The Regents of The University of California, Oakland, CA (US)

(21) Appl. No.: 17/863,149

(22) Filed: Jul. 12, 2022

Related U.S. Application Data
(63) Continuation of application No. PCT/US2021/013220, filed on Jan. 13, 2021.
(60) Provisional application No. 62/960,496, filed on Jan. 13, 2020.

(51) Int. Cl. C07K 16/10; C07K 16/28

(52) U.S. Cl. CPC C07K 16/1063; C07K 16/2866; C07K 16/2809

(57) ABSTRACT

Provided herein are methods for preventing or treating a human immunodeficiency virus (HIV) infection or a simian immunodeficiency virus (SIV) infection in a subject. The methods include administering to the subject (a) a reservoir-depleting agent that binds to a host protein on a reservoir cell, and (b) an antiviral vaccine.

### CROSS-REFERENCE TO RELATED APPLICATIONS

This application is a continuation of International Application No. PCT/US2021/013220, filed Jan. 13, 2021, which claims priority to U.S. Provisional Application No. 62/960,496, filed Jan. 13, 2020, the disclosures of which are hereby incorporated by reference in their entirety for all purposes.

### BACKGROUND OF THE INVENTION

Rhesus cytomegalovirus (RhCMV)-vectored vaccines control early HIV/SIV infection based on their ability to elicit high frequency, effector-differentiated, broadly targeted virus-specific T cells in potential sites of early viral replication, where they prevent the establishment of a persistent reservoir. First-generation RhCMV/SIV vaccines used prophylactically protect ˜50% of vaccinated monkeys via elimination of the nascent infection. Unfortunately, the vaccines seem to have only limited efficacy when used therapeutically, i.e., in the presence of an established HIV/SIV reservoir. While a prophylactic HIV vaccine with 50% efficacy would be beneficial, a core goal in the field must be to provide a therapeutic vaccine, benefitting infected individuals and potentially enabling HIV cure. Thus, there is a need for improved therapies for treating HIV infections.

### BRIEF SUMMARY OF THE INVENTION

In one aspect, the present disclosure provides a method for preventing or treating a human immunodeficiency virus (HIV) infection or a simian immunodeficiency virus (SIV) infection in a subject, the method comprising administering to the subject (a) a reservoir-depleting agent that binds to a host protein on a reservoir cell, and (b) one or more antiviral vaccines. In some embodiments, the reservoir cell is a CCR5⁺ cell, a CD4⁺ cell, and/or a CD8⁺ cell.

In some embodiments, the host protein is a protein on the surface of a reservoir cell. In some embodiments, the host protein can be a cell surface receptor of a reservoir cell. In certain embodiments, the host protein is CCR5. In some embodiments, the host protein is CD3. In some embodiments, the host protein is CD4. In yet other embodiments, the host protein is CD8.

In some embodiments, the reservoir-depleting agent depletes CCR5⁺ cells. In some embodiments, the reservoir-depleting agent depletes CD4⁺ cells (e.g., CCR5⁺ CD4⁺ cells). In some embodiments, the reservoir-depleting agent depletes CD8⁺ cells (e.g., CCR5⁺ CD8⁺ cells).

In certain embodiments, the reservoir-depleting agent depletes CCR5⁺ cells, CD4⁺ cells, and/or CD8⁺ cells in the GI tract, lymph node, spleen, thymus, or lumbar spinal cord (e.g., the GI tract or lymph node).

In some embodiments, the reservoir-depleting agent is an antibody. For example, the antibody can be an antibody that binds to CD4 or an antibody that binds to CCR5. In some embodiments, the antibody is a bispecific antibody (e.g., a bispecific antibody that binds to CCR5 and CD3).

In some embodiments, the reservoir-depleting agent is an immunotoxin comprising a CCR5 ligand and a toxin. In certain embodiments, the CCR5 ligand is selected from the group consisting of RANTE5/CCL5, MIP-1alpha/CCL3, MIP-1beta/CCL4, CCL3L1, and CCL4L1. In certain embodiments, the toxin comprises part or all of a protein selected from the group consisting of diphtheria toxin, Pseudomonas exotoxin, ricin, gelonin, and saponin. In some embodiments, the toxin is selected from the group consisting of DT385, DT388, DT390, DAB389, DAB486, PE35, PE38, and PE40. The toxin can be modified to prevent cell entry independent of the CCR5 ligand, to reduce immunogenicity, to improve target-cell toxicity, or to reduce untargeted toxicity. Examples of modifications to toxins can be found in, e.g., Zhu et al., Biomed Res Int. 2017: 7929286.

In some embodiments of this aspect, the reservoir-depleting agent can simultaneously bind two target host molecules. For example, the reservoir-depleting agent can comprise fused variable domains of immunoglobulin heavy chains and light chains. The reservoir-depleting agent can be a bispecific T-cell engager, a DART, or a tandem diabody. In specific embodiments, the reservoir-depleting agent binds to CCR5 and CD3.

The antiviral vaccine in the methods described herein can be a cytomegalovirus-vectored vaccine, a modified vaccinia ankara B-vectored (MVA-B-vectored) vaccine, a gp120 envelope protein, a gp160 envelope protein, a recombinant adenovirus-5 HIV vaccine, a recombinant adenovirus-26 HIV vaccine, a recombinant adenovirus-35 HIV vaccine, a recombinant simian adenovirus HIV vaccine (e.g., chimp or gorilla adenovirus), a killed whole-HIV-1 vaccine (SAV001), or a canarypox vector. In some embodiments, the same antiviral vaccine is serially delivered. In other embodiments, two or more different antiviral vaccines are serially delivered.

In methods described herein, in some embodiments, the subject is further administered an antiretroviral therapy (ART). In some embodiments, the ART comprises tenofovir, emtricitabine, and/or dolutegravir.

In some embodiments, the reservoir-depleting agent is administered before the antiviral vaccine. For example, the reservoir-depleting agent is administered at least one week before the antiviral vaccine. In some embodiments, the reservoir-depleting agent is administered after the antiviral vaccine. For example, the reservoir-depleting agent is administered at least one week after the antiviral vaccine. In other embodiments, the reservoir-depleting agent and the antiviral vaccine are administered substantially at the same time.

In some embodiments of the methods, multiple doses of the antiviral vaccine are administered. In certain embodiments, the same antiviral vaccine is administered in multiple doses. In other embodiments, two or more different antiviral vaccines are administered in multiple doses.

In some embodiments, the reservoir-depleting agent is administered within 21 days (e.g., within 1, 2, 3, 4, 5, 6, 7, 8, 9, 10, 11, 12, 13, 14, 15, 16, 17, 18, 19, or 20 days) after the subject is exposed to an HIV or an SIV. In some embodiments, the antiviral vaccine is administered within 21 days (e.g., within 1, 2, 3, 4, 5, 6, 7, 8, 9, 10, 11, 12, 13, 14, 15, 16, 17, 18, 19, or 20 days) after the subject is exposed to an HIV or an SIV.

In some embodiments, the subject is administered the ART before being administered the reservoir-depleting agent or the antiviral vaccine. In certain embodiments, the subject is administered the ART during the entire duration of being administered the reservoir-depleting agent and the antiviral vaccine.

In some embodiments, the subject is a primate (e.g., a human or a simian).

Other objects, features, and advantages of the present disclosure will be apparent to one of skill in the art from the following detailed description and figures.

### BRIEF DESCRIPTION OF THE DRAWINGS

FIGS. 1A-1F: Lymphopenia and subsequent CCR5 depletion in blood post CCR5xCD3 bsAb administration. FIG. 1A: Flow cytometry of whole blood from 1.5-year old rhesus macaques treated with 1 mg/kg bsAb, gated on FSC/SSC, single cells, and live/dead only. Blood was taken prior to treatment, 4 h after treatment, and 1 w after treatment. FIGS. 1B and 1C: Representative cytograms (B) and longitudinal plots (C) of CD3⁺ lymphocytes from macaques receiving 1 mg/kg at pre-treatment, 4 h, 1 w, and 3 w from drug administration. FIG. 1D: Flow cytometry of whole blood from 1.5-year old rhesus macaques treated with 3 mg/kg bsAb. FIGS. 1E and 1F: Representative cytograms (E) and longitudinal plots (F) of CD3⁺ lymphocytes from rhesus macaques receiving 3 mg/kg antibody.

FIGS. 2A-2I: Depletion of CCR5⁺ lymphocytes from colon and lymph node after bsAb. FIG. 2A: Flow cytograms of colonic CD3⁺ lamina propria lymphocytes in two 1.5-year old macaques receiving 1 mg/kg bsAb. The top row demonstrates nearly complete depletion in one of these two animals (84% and 93% of CD4⁺ and CD4⁻ cells, respectively). FIGS. 2B and 2C: Essentially complete depletion of colonic CCR5⁺ cells among CD3⁺ lamina propria lymphocytes from 1.5-year old macaques receiving 3 mg/kg antibody. Representative cytograms (B) show 96% depletion of CD8⁺ T cells expressing CCR5. FIGS. 2D and 2E: Depletion of CCR5⁺ cells from among lymph node CD3⁺ lymphocytes of 1.5-year old macaques receiving 1 mg/kg (D) or 3 mg/kg (E) antibody. FIGS. 2F-2I: Depletion of CCR5⁺ cells from among colon CD3⁺CD4⁺ (F), colon CD3⁺CD8⁺ (G), or lymph node CD3⁺(I) cells of 8-month-old rhesus macaques receiving 3 mg/kg bispecific antibody. A graph of the extent of depletion in colon is shown in H.

FIGS. 3A-3C: CD4 and CCR5 depletion are associated with more rapid viral-load decay under antiretroviral therapy (ART). FIG. 3A: Experimental design: group A is negative control, group B is anti-CD4, and group C is bsAb. FIG. 3B: Inspection of viral-load traces in the period before ART is withdrawn suggests more rapid control of SV after treatment with anti-CD4 or anti-CD3/CCR5 bsAb. FIG. 3C: Slope of viral-load decay in groups A-C in logs per day, between peak viral load and three weeks later. Decay is significantly more rapid in both groups B and C, when correcting for the presence of controller alleles (p=0.012 and 0.039, respectively).

FIG. 4: Cure of most early-treated infants using CD3/CCR5 bsAb. Cure is indicated by failure to detect the virus after withdrawal of ART in 1/8 CD4R1-treated animals (middle panel) and 4/7 CD3/CCR5 bsAb-treated animals (right panel).

FIGS. 5A-5D: Frequency of CCR5⁺ cells among infant macaques in the first year of life. FIG. 5A: Frequency of CCR5⁺ cells among all circulating CD4⁺ T cells, shown over the first year of life in 35 rhesus macaque infants. FIG. 5B: Frequency of CCR5⁺ cells among CD4⁺ memory T cells (CD95⁺). FIG. 5C: Frequency of CCR5⁺ cells from 5-12 months of age among all CD4⁺ T cells, naive cells, central memory cells, or effector memory cells. FIG. 5D: Frequency of CCR5⁺ cells from 5-12 months of age among CD8⁺ T cell subsets.

FIG. 6: An exemplary timeline showing a combined therapeutic regimen.

### DETAILED DESCRIPTION OF THE INVENTION

### I. INTRODUCTION

HIV is extraordinarily difficult to eradicate from the body due to integration of its genome into reservoir cells. The integrated genome can remain latent for an indefinite period without gene expression—resulting in a host cell that remains invisible to the host immune system and can therefore persist indefinitely. When HIV-infected people stop taking antiretroviral drugs (antiretroviral therapy (ART), i.e., drugs targeting viral proteins), the virus invariably emerges from these cellular reservoirs. Furthermore, the number of such reservoir cells is thought to affect the rapidity of viral rebound, that is, the speed with which HIV emerges from latency and establishes robust replication that is evident in blood and tissues.

Many approaches have been tried to eliminate HIV from infected people, with very little success. Therapeutic vaccines stimulate the immune system of an infected person to respond to the virus, in hope that the new immune responses can prevent rebound by attacking either the virus itself (with antibodies) or newly infected cells. No vaccine has ever been shown conclusively to be effective for therapy of HIV infection, or for therapy of SIV, the non-human primate equivalent. Latency-reversing agents (LRAs) aim to prevent silencing of the integrated SIV genome, with the aim of exposing all infected cells to the immune system. Epigenetic silencing of HIV (also known as “block and lock”) attempts the opposite, i.e., to assure continued silence of the viral genome and prevent its spread within the body after ART drugs are withdrawn.

The present disclosure is directed to a combined therapeutic regimen comprising: (i) a vaccine with efficacy before infection (prophylactic vaccines) or in early infection (therapeutic vaccines), when HIV reservoir levels are low; and (ii) a second agent that targets host HIV-reservoir cells for depletion. The key insights are that some vaccines used for prophylaxis act, not by completely preventing HIV infection, but by promoting host immune responses that control a nascent infection driven by a small pool of infected cells; that reservoir-depletion strategies targeting subsets of host cells can be remarkably effective in reducing the pool of reservoir cells available to drive viral rebound; and that such vaccines and reservoir-depleting agents work synergistically in a combined therapeutic setting to provide sustained reduction of the virus in the body, or elimination of the virus.

As described in detail below, in preliminary experiments testing a host HIV reservoir-targeting agent alone, we cured most early-treated newborns (4/7 animals starting ART seven days post infection) using a single injection of an anti-CD3/CCR5 bispecific antibody (bsAb). The cured animals remained aviremic for up to 200 days following ART withdrawal, even after CD8 depletion to encourage viral rebound. Sensitive viral outgrowth assays failed to recover replication-competent virus, as indicated by absence of cytopathic effect for CEMx174 cells. Thus, results obtained so far show that these animals have achieved at least “functional” and perhaps sterilizing cure.

This startling result has a number of important ramifications. It indicates the possibility of cure for some fraction of the ˜160,000 infants and children infected with HIV annually, if the infection can be detected within one week. More importantly, the experiment demonstrates for the first time that this reservoir-targeting agent successfully reduces the number of reservoir cells that can produce virus. We discovered that such a reservoir-depleting agent is particularly suitable for administration to chronically HIV-infected people (weeks to years) in combination with a vaccine capable of suppressing early, nascent infection. CMV-vectored vaccines are one example, as described below.

### II. DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values ×, 0.8×, 0.81×, 0.82×, 0.83×, 0.84×, 0.85×, 0.86×, 0.87×, 0.88×, 0.89×, 0.9×, 0.91×, 0.92×, 0.93×, 0.94×, 0.95×, 0.96×, 0.97×, 0.98×, 0.99×, 1.01×, 1.02×, 1.03×, 1.04×, 1.05×, 1.06×, 1.07×, 1.08×, 1.09×, 1.1×, 1.11×, 1.12×, 1.13×, 1.14×, 1.15×, 1.16×, 1.17×, 1.18×, 1.19×, and 1.2×. Thus, “about ×” is intended to teach and provide written description support for a claim limitation of, e.g., “0.98×.”

The term “reservoir-depleting agent” refers to an agent that binds to a component of a cell that can be infected with a virus, e.g., SIV or HIV. For example, the reservoir-depleting agent can bind to a cell surface receptor of a cell type that can be infected with the virus. In some embodiments, the reservoir-depleting agent can bind to a cell surface receptor of a T cell that can be infected with a virus, e.g., SIV or HIV. In some embodiments, the reservoir-depleting agent can be an antibody or an immunotoxin. In certain embodiments, the reservoir-depleting agent can be an anti-HIV neutralizing antibody.

The term “reservoir cell” refers to a cell that can be infected with a virus, e.g., SIV or HIV, for example, a immune cell (e.g., a T cell) of an infected subject.

The term “host protein” refers to a protein on the surface of a reservoir cell. In some embodiments, the host protein can be a cell surface receptor of a reservoir cell. In certain embodiments, the host protein is CCR5 . In some embodiments, the host protein is CD3. In some embodiments, the host protein is CD4. In yet other embodiments, the host protein is CD8.

The term “immunotoxin” refers to a protein containing a targeting portion that is linked to a toxin. In some embodiments, when the immunotoxin binds to a cell, e.g., a cell that is infected with a virus, the immunotoxin is taken in through endocytosis, and the toxin then kills the cell. The targeting portion of the immunotoxin can be an antibody or a ligand.

The term “antiviral vaccine” refers to a drug that can protect subjects who do not have a viral infection (a preventive/prophylactic vaccine), or treat a virus-infected subject (a therapeutic vaccine). In the case of an HIV infection, the antiviral vaccine can be an HIV vaccine. For example, a preventative HIV vaccine can be given to subjects who are not infected with an HIV. A therapeutic HIV vaccine can be given to subjects who are infected with an HIV but who do not yet have any symptoms of an HIV infection (i.e., the HIV is latent in the subject) or to subjects who are infected with an HIV and show symptoms of an HIV infection (i.e., the HIV is active in the subject).

The terms “antiretroviral therapy” or “ART” refer to a combination of HIV medicines (called an HIV regimen) to treat an HIV infection. A person's initial HIV regimen generally includes three antiretroviral drugs from at least two different HIV drug classes.

The term “substantially at the same time” refers to a reservoir-depleting agent and an antiviral vaccine being administered to a subject within one day (e.g., within 1 hour, within 5 hours, within 10 hours, within 15 hours, within 20 hours, or within 24 hours) of each other.

The terms “polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “subject,” “individual,” and “patient” are used interchangeably herein to refer to a vertebrate, preferably a mammal, more preferably a human. Mammals include, but are not limited to, murines, mice, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoral, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, depot formulations, etc.

The term “treating” refers to an approach for obtaining beneficial or desired results including, but not limited to, a therapeutic benefit and/or a prophylactic benefit. “Therapeutic benefit” means any therapeutically relevant improvement in or effect on one or more diseases, conditions, or symptoms under treatment. Therapeutic benefit can also mean to effect a cure of one or more diseases, conditions, or symptoms under treatment. Furthermore, therapeutic benefit can also mean to increase survival. For prophylactic benefit, the compositions may be administered to a subject at risk of developing a particular disease, condition, or symptom, or to a subject reporting one or more of the physiological symptoms of a disease, even though the disease, condition, or symptom may not yet be present.

### III. METHODS FOR PREVENTING OR TREATING AN HIV INFECTION OR AN SIV INFECTION

Provided herein are methods for preventing or treating a human immunodeficiency virus (HIV) infection or a simian immunodeficiency virus (SIV) infection in a subject, the method comprising administering to the subject (a) a reservoir-depleting agent, and (b) an antiviral vaccine. The inventors have discovered a combined therapeutic regimen containing a reservoir-depleting agent that targets a host protein on infected cells to reduce the pool of reservoir cells available to drive viral rebound and an antiviral vaccine that promotes host immune responses to control nascent infections driven by infected cells. A host protein on infected cells can be a cell surface receptor. In certain embodiments, the host protein is CCR5. In some embodiments, the host protein is CD3. In some embodiments, the host protein is CD4. In yet other embodiments, the host protein is CD8.

In some embodiments, the reservoir-depleting agent depletes CD4⁺ cells. In some embodiments, the reservoir-depleting agent that depletes CD4⁺ cells is auranofin. In some embodiments, the reservoir-depleting agent depletes CCR5⁺ cells. In particular embodiments, the reservoir-depleting agent binds to a C-C chemokine receptor type 5 (CCR5) receptor. In some embodiments of the methods, the subject is also administered an ART. The ART can be administered before the start of either the reservoir-depleting agent or the antiviral vaccine. In some embodiments, the ART can be administered to the subject throughout the entire duration of the reservoir-depleting agent and the antiviral vaccine administration. In some embodiments, the ART is withdrawn after administration of the reservoir-depleting agent and the antiviral vaccine.

CCR5 plays an essential role in HIV pathogenesis as the main coreceptor used by macrophage (M)-tropic strains of human HIV-type 1 (HIV-1) and HIV-type 2 (HIV-2), which are the strains primarily responsible for viral transmission from one host to another. In methods described herein, a reservoir-depleting agent that binds to CCR5 is used to deplete CCR5⁺ cells. In some embodiments, a reservoir-depleting agent is an antibody, e.g., an antibody that binds to CCR5. In certain embodiments, the reservoir-depleting agent is a bispecific antibody, e.g., a bispecific antibody that binds to CCR5 and CD3. As demonstrated herein, a bispecific antibody that binds to CCR5 and CD3 was able to deplete CCR5⁺ cells from the the blood of eight-month-old and 1.5-year-old macaques.

In other embodiments, a reservoir-depleting agent is an immunotoxin comprising a CCR5 ligand and a toxin. In certain embodiments, the CCR5 ligand is selected from the group consisting of RANTES/CCL5, MIP-1alpha/CCL3, MIP-1beta/CCL4, CCL3L1, and CCL4L1. In certain embodiments, the toxin comprises part or all of a protein selected from the group consisting of diphtheria toxin, Pseudomonas exotoxin, ricin, gelonin, and saponin. In some embodiments, the toxin is selected from the group consisting of DT385, DT388, DT390, DAB389, DAB486, PE35, PE38, and PE40. The toxin can be modified to prevent cell entry independent of the CCR5 ligand, to reduce immunogenicity, to improve target-cell toxicity, or to reduce untargeted toxicity. Examples of modifications to toxins can be found in, e.g., Zhu et al., Biomed Res Int. 2017: 7929286.

In certain embodiments, the reservoir-depleting agent can be an anti-HIV neutralizing antibody.

In some embodiments of the methods, the reservoir-depleting agent depletes CD4⁺ cells (e.g., CCR5⁺ CD4⁺ cells) and/or CD8⁺ cells (e.g., CCR5⁺ CD8⁺ cells). The inventors also showed that a bispecific antibody to CCR5 and CD3 was able to efficiently deplete CCR5⁺ cells in tissues that are difficult to penetrate, such as the lymph node. In certain embodiments, the reservoir-depleting agent depletes CCR5⁺ cells, CD4⁺ cells, and/or CD8⁺ cells in the GI tract, lymph node, spleen, thymus, or lumbar spinal cord of the subject.

The methods described herein include the combined therapy of a reservoir-depleting agent and an antiviral vaccine. Multiple HIV vaccines are being tested in various stages of clinical trials. Examples of an antiviral vaccine that can be used in the methods described herein include, but are not limited to, a cytomegalovirus-vectored vaccine, a modified vaccinia ankara B-vectored (MVA-B-vectored) vaccine, a gp120 envelope protein, a gp160 envelope protein, a recombinant adenovirus-5 HIV vaccine, a recombinant adenovirus-26 HIV vaccine, a recombinant adenovirus-35 HIV vaccine, a recombinant simian adenovirus HIV vaccine (e.g., chimp or gorilla adenovirus), a killed whole-HIV-1 vaccine (SAV001), and a canarypox vector. Other examples of antiviral vaccines are described in, e.g., Vekemans et al., Lancet HIV. S2352-3018(19)30294-2; 2019; and Bekker et al., Lancet. S0140-6736(19)32682-0, 2019, which are incorporated herein by reference in their entireties. In some embodiments of the methods described herein, wherein multiple doses of the antiviral vaccine are administered to the subject.

In the combined therapy described herein, in some embodiments, the reservoir-depleting agent is administered before the antiviral vaccine, e.g., at least one week (e.g., at least 2, 4, 6, 8, 10, 12, 14, 16, 18, 20, 22, or 24 weeks) before the antiviral vaccine. In other embodiments, the reservoir-depleting agent is administered after the antiviral vaccine, e.g., at least one week (e.g., at least 2, 4, 6, 8, 10, 12, 14, 16, 18, 20, 22, 24, 26, 28, 30, 32, or 34 weeks) after the antiviral vaccine. In other embodiments, the reservoir-depleting agent and the antiviral vaccine can be administered substantially at the same time, i.e., administered within about 1, 2, 3, 4, 5, 6, 7, 8, 9, 10, 11, 12, 13, 14, 15, 16, 17, 18, 19, 20, 21, 22, 23, or 24 hours of each other. In the combined therapy described herein, in some embodiments, the reservoir-depleting agent is administered before the antiviral vaccine, e.g., within 6 days (e.g., within 1, 2, 3, 4, or 5 days) before the antiviral vaccine. In other embodiments, the reservoir-depleting agent is administered after the antiviral vaccine, e.g., within 6 days (e.g., within 1, 2, 3, 4, or 5 days) after the antiviral vaccine.

In some embodiments of the methods, the reservoir-depleting agent and/or the antiviral vaccine are administered to the subject within 21 days (e.g., within 1, 2, 3, 4, 5, 6, 7, 8, 9, 10, 11, 12, 13, 14, 15, 16, 17, 18, 19, or 20 days) after the subject is exposed to an HIV or a SIV.

### IV. KITS

Provided herein are also kits for treating an HIV infection or an SIV infection. In some embodiments, the kit comprises a reservoir-depleting agent (e.g., an antibody (e.g., a bispecific anti-CCR5/CD3 antibody) or an immunotoxin) described herein and an antiviral vaccine described herein. In some embodiments, the kit also includes drugs used in an ART.

Kits of the present disclosure can be packaged in a way that allows for safe or convenient storage or use (e.g., in a box or other container having a lid). Typically, kits of the present disclosure include one or more containers, each container storing a particular kit component such as a reservoir-depleting agent (e.g., an antibody (e.g., a bispecific anti-CCR5/CD3 antibody) or an immunotoxin), and an antiviral vaccine, and so on. The choice of container will depend on the particular form of its contents, e.g., a kit component that is in liquid form, powder form, etc. Furthermore, containers can be made of materials that are designed to maximize the shelf-life of the kit components. As a non-limiting example, kit components that are light-sensitive can be stored in containers that are opaque.

In some embodiments, the kit contains one or more reagents. In some instances, the reagents are useful for preparing the reservoir-depleting agent (e.g., an antibody (e.g., a bispecific anti-CCR5/CD3 antibody) or an immunotoxin) and/or the antiviral vaccine for administration to a subject (e.g., pharmaceutically acceptable carriers). In some embodiments, the kit contains paraphernalia for administering the reservoir-depleting agent and/or the antiviral vaccine to the subject (e.g., syringes, needles, vials), obtaining a sample from a subject (e.g., blood tubes or other biofluid tubes, syringes, disposable equipment for preparing a venipuncture site), or processing a sample obtained from a subject (e.g., test tubes, slides). In yet other embodiments, the kit further contains instructions for use.

### V. EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Depletion of the CCR5⁺ Reservoir Under Antiretroviral Therapy (ART) Coverage

### Specific Aims

The goal of this project is to cure SIV-infected and early-treated infant macaques via transient depletion of the CCR5⁺ reservoir under antiretroviral therapy (ART) coverage. In experiments completed previously, we cured most early-treated newborns (4/7 animals starting ART seven days post infection) using a single injection of anti-CD3/CCR5 bispecific antibody (bsAb). The cured animals remained aviremic for up to 200 days following ART withdrawal, even after CD8 depletion to encourage viral rebound. No proviral DNA was detected in circulating cells at any time point following ART withdrawal. Sensitive viral outgrowth assays failed to recover replication-competent virus. Thus, results obtained show that these animals have achieved at least “functional” and apparently sterilizing cure.

This result has a number of important ramifications. It suggests the possibility of cure for some fraction of the ˜160,000 infants and children infected with HIV annually. More importantly, the experiment demonstrates for the first time that one-shot, pharmacologic cure of an established SIV infection is possible—without the need for extreme measures such as bone-marrow transplant. Cures may also be achievable in older individuals or in those starting ART treatment later. More plausibly, perhaps, depletion of CCR5⁺ cells may prove to be a critical component of combination strategies for HIV cure.

The bsAb we employed achieves very efficient, transient depletion of CCR5⁺ cells. The therapy also causes an inflammatory reaction with cytokine production and temporary CD3⁺ lymphopenia. Our specific aims are:

Specific Aim 1. Optimize reagents for CCR5 depletion without T-cell activation. Here we prepare and test, in four sub-aims, new reagents that are free of contaminants and/or inherently not activating: 1a) Prepare bsAb free of parental anti-CD3 antibody by purification on a light chain-specific affinity column; 1b) Produce optimized immunotoxins based on CCR5 ligand-toxin fusions; 1c) Optimize bsAb for CCR5 affinity using new anti-CCR5 arms; and 1d) Test pharmacokinetics and pharmacodynamics of candidate agents in vivo, with a focus on CCR5 depletion from gut tissues without immune activation.

Specific Aim 2. Test if T-cell activation contributes to SIV cure achieved in newborn macaques. Here we test three new agents, selected in sub-aim 1d, for capacity to cure early-treated newborn macaques: (A) purified bsAb lacking parental anti-CD3; (B) one immunotoxin; and (C) one optimized bsAb with superior CCR5 binding, also free of parental anti-CD3.

Specific Aim 3. Determine if recipient age or reservoir maturity limit SIV cure by CCR5 reservoir depletion. Older macaques often have a larger population of CCR5⁺ cells. They may also have a more competent immune system that is more capable of contributing to cure. In this aim we test both possibilities using an optimized agent from Specific Aim 2, which cures early-treated newborn macaques while causing minimal immune activation. In this aim, we: 3a) Evaluate frequency of cure in juvenile macaques; and 3b) Evaluate frequency of cure when treatment is initiated 14 days after infection.

This research will determine if the CCR5⁺ reservoir is an “Achilles' heel” in early SIV and HIV infection. In addition to key questions about efficacy, we will be interested to learn if the host immune system contributes to SIV elimination, as might be suggested by demonstration of immune responses in cured animals, or by the finding that older animals with presumably more robust immunity are cured more easily. Demonstration of a contribution from host immunity supports the combined use of CCR5 depletion and therapeutic vaccination for treatment of HIV-infected people.

### Significance

RhCMV/SIV Vaccines Impair Viral Replication at the Earliest Stage, at Viral Entry Sites and before Dissemination.

A number of experiments suggested that HIV and SIV infections might be vulnerable to immune control or pharmacologic clearance in the first hours to days of infection, before establishment of the reservoir cells that sustain infection. Rhesus cytomegalovirus (RhCMV)-vectored vaccines were designed to exploit this possible window of vulnerability, based on their ability to elicit virus-specific T cells that are localized to reservoir sites. Indeed, the pattern of protection observed in approximately 50% of RhCMV/SIV vector-vaccinated rhesus macaques after intrarectal SIVmac239 challenge was consistent with immunologic interception of the nascent SIV reservoir at the portal of viral entry, before systemic spread. Protected macaques manifested transient viremia at the onset of infection, followed by control of plasma SIV levels to below the level of detection, except for occasional plasma viral ‘blips’ that waned over time, and at necropsy demonstrated only trace levels of tissue-associated SIV RNA and DNA using ultrasensitive assays. The occurrence of plasma viral blips and “breakthrough” progressive infection observed in 1 of 13 RhCMV/SIV vector-protected macaques 77 days after infection indicated that SIV was not immediately cleared from protected animals. Nonetheless, failure to find more than trace levels of SIV nucleic acid in systemic lymphoid tissues is consistent with predominant containment to the portal of entry.

Lengthy Remission of HIV Infection can Occur with Treatment soon after Infection

Remission of an HIV-infected infant was described (Persaud, D. Functional HIV Cure after Very Early Antiretroviral Therapy in an Infected Infant. in 20th Conference on Retroviruses and Opportunistic Infections. 2013. Atlanta, Ga.: 20th Conference on Retroviruses and Opportunistic Infections). The infant was born to an infected mother and started on ART at 31 hours of age while blood samples were tested to assess infection status. Test results from two independent blood samples subsequently confirmed the presence of HIV DNA and RNA, fulfilling diagnostic criteria for HIV infection in perinatally-exposed infants. Viral load steadily declined during the next 28 days on ART, became undetectable at one month, and remained so through 18 months of age, when the infant stopped ART. At 23 months of age her HIV-RNA was still undetectable despite the absence of any ART, and HIV DNA was undetectable as well. At 26 months of age proviral DNA was detected at the level of 4.2 copies per million PBMC. No replication-competent virus was recovered from culture of 22×10⁶ resting CD4⁺ T cells. Neither virus-specific T cells nor antibodies were detected. Unfortunately, viremia suddenly rebounded in this child 27 months after treatment interruption.

14 cases of apparent functional cure in adults were reported (Saez-Cirion et al., PLoS Pathog, 9(3):p. e1003211, 2013). These subjects started therapy an average of 1.6 months after initial exposure to HIV and continued therapy for 12 to 92 months (median, 36.5 months). Following cessation of therapy, viral load remained under 400 copies/mL for at least 24 months but usually much longer (median, 89 months). PBMC-associated HIV DNA levels in functionally cured adults were similar to those seen in HIV controllers and much lower than in patients with uncontrolled infection or who had started treatment in the chronic phase. Of eight patients for whom longitudinal HIV DNA levels were available after treatment interruption, five demonstrated progressive decline in HIV DNA levels over the years. To explain this decline, the authors of the study measured HIV DNA content among sorted CD4⁺ T cell subsets. They found that long-lived cells such as naíve and central memory cells contributed very minimally to the cured individuals' total HIV reservoir in resting CD4⁺ T cells, which might have contributed to the gradual shrinking of the reservoir while off treatment.

A major goal of experiments was to use reservoir depletion to re-create the lengthy remission. The hypothesis was that shrinking the reservoir might enable such remission, given the clues suggested by the data in Saez-Cirion et al., supra. The data we present below suggests that this aim of achieving, at least, lengthy remission, was achieved.

Establishment of the Viral Reservoir by Three Days after Intrarectal SIV Infection; Failure to Cure in all Cases when Treatment is Initiated at Seven Days.

It was demonstrated that the viral reservoir is seeded by 3 days after intrarectal SIV infection of young adult rhesus macaques (see, Whitney et al., Nature, 512(7512):p. 74-7, 2014). These findings fit nicely with data showing that post-exposure prophylaxis (PEP) is ineffective beyond 2-3 days post infection: successful PEP may be impossible after establishment of the long-lived reservoir (or, at least, in this situation “prophylaxis” would be an inappropriate label). Functional cures, in contrast, are associated by definition with persistence of HIV DNA in reservoir cells. Importantly, these authors found no instance of apparent cure when triple ART therapy was started at 3 days or 7 days after infection (0/8 total).

A more nuanced and better-powered study of animals was conducted where the animals were infected intravenously with only two focus-forming units of SIVmac239—a choice that might be imagined to result in slow spread of the virus and excellent likelihood of cure, if possible using ART alone. A total of 39 animals were treated at times from 4-12 days after infection, which was detected using a novel immune biomarker (CD169 expression on monocytes) and confirmed by demonstration of clear viremia in all cases. In this study, as well, no instance of cure was demonstrated when ART was started 7 days after infection (0/10). Furthermore, only one instance of apparent cure was demonstrated among 19 animals treated 4-6 days after infection.

These careful studies demonstrated conclusively that cure is extremely rare when ART is initiated 7 days after infection. Taking both studies into account it would appear that the frequency of cure is at most ˜1/37 (2.7%)—reflecting the single animal exhibiting control among 37 treated in both studies at 7 days or earlier.

We show below that depletion of host HIV reservoir cells during ART can facilitate “functional cure” of infant macaques treated soon after infection.

Modest Restriction of the T_{CM} Compartment Induces Containment of Viral Load Following ART Suspension.

The mechanism of action of the gold-based compound auranofin is only partly understood. However, the drug is toxic to T cells: auranofin impairs the proliferative capacity of T cells in vitro, decreases production of pro-inflammatory cytokines in macrophages and T cells, and induces apoptosis in the Jurkat T-cell line. It was demonstrated that auranofin treatment of macaques induced an —30% reduction in the frequency of the long-lived T_{CM}/T_{TM} CD4⁺ T cell subpopulation in peripheral blood, accompanied by a relative increase in the TEM subset, whereas the frequency of naive cells remained unchanged. In a small study of five SIV-infected animals, the combination of auranofin and intensified ART (iART) significantly reduced the CD4⁺ T-cell-associated vDNA in SIVmac251-infected macaques, suggesting that memory CD4⁺ T cells harboring virus were killed by auranofin. In two of these animals, viral loads did not rebound until weeks 7 and 8 following therapy suspension, a time longer than was observed in iART-alone controls (mean 1.5 weeks). Furthermore, although early peaks reminiscent of new acute infections were observed, viral loads of auranofin-treated monkeys dropped to set points that were lower than either the pre-ART viral loads from the same animals or the viral set points of iART-only controls.

Thus, in this small experiment and a subsequent followup, a mere 30% reduction in availability of T_{CM} and T_{TM} cells was associated with a significant delay in viral rebound after cessation of therapy. This overall effect in the T_{TM} compartment was presumably associated with a similar magnitude of effect on CCR5⁺ T_{CM} cells. We achieve far better reductions (at least 75%) in those specific segments of the T_{CM} and T_{EM} that are vulnerable to infection (i.e., CCR5⁺ T_{CM} and T_{EM}, in addition to macrophages and other CCR5⁺ cells). Furthermore, our inventive contribution is to combine reservoir-depleting agents such as these with therapeutic vaccines, which provide the extraordinary T-cell response necessary to provide continuous control of the infection.

### Innovation

Novelty of this Approach to Reduction of Viral Reservoirs.

The field has made little progress on direct reservoir depletion as an approach to HIV cure, with greater focus having been placed on broadly neutralizing antibodies (bNAbs) and latency reactivating agents (LRAs). One reason for this focus might be the lack of selective agents for reservoir depletion—that is, agents affecting only cells that reveal themselves in some way to be definitely infected. We suggested instead that relatively non-specific depletion, e.g., of CD4⁺ T cells, is a reasonable approach to cure, especially when the depletion is transient and the cure long lasting. CCR5 depletion should be seen as partially selective, because most infected cells are CCR5⁺, even though the vast majority of CCR5⁺ cells are not infected. Depletion of CCR5 -expressing cells has the additional advantage that even a complete, lifelong lack of CCR5 expression has a limited deleterious effect; transient depletion might therefore be relatively harmless.

Others have proposed more specific methods for depletion of cells thought to harbor HIV, particularly memory CD4⁺ T cells, e.g., auranofin. These methods have great promise and also intellectual appeal. However, there are few reliable tools available for specific depletion of CD4⁺ T cell subsets from humans or non-human primates. More importantly, it is known already that HIV is harbored in naïve CD4⁺ T cells and CD4⁺ macrophages, neither of which would be eliminated by compounds targeting only memory CD4⁺ T cells.

### Important Focus on Newborns.

It is true that infection is more likely to be recognized early among infants, permitting cure to be attempted, and that every success will provide more years of healthy living.

Durable remission of HIV and SIV infections in newborn macaques and humans is possible under the right circumstances. The therapy described herein can also apply to older individuals and/or to those whose infections are recognized later.

### Example 2. Anti-CD3/CCR5 Bispecific Antibody Depletes CCR5⁺ Cells from Blood in Vivo

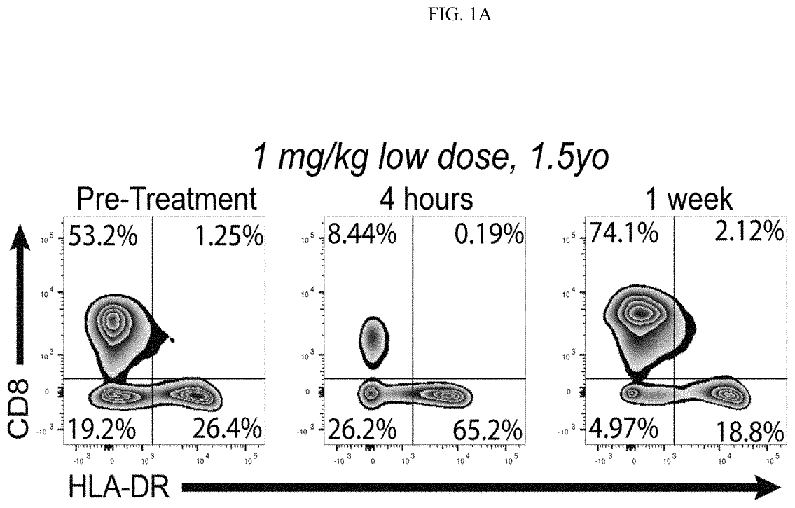

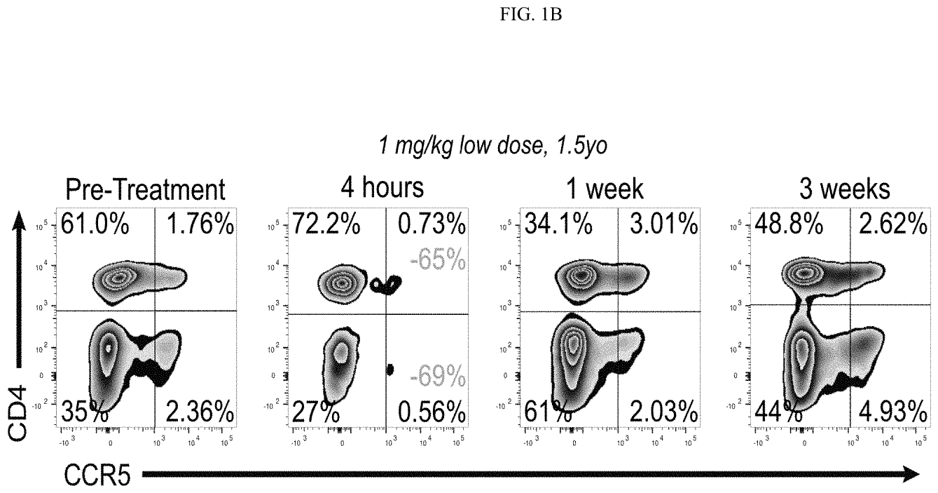

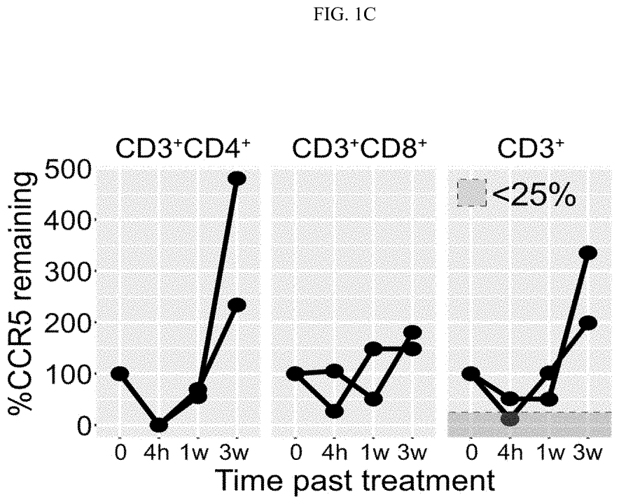

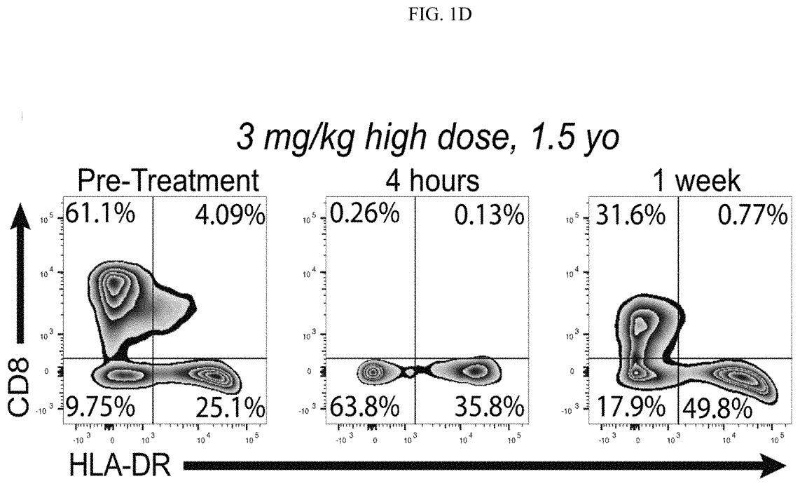

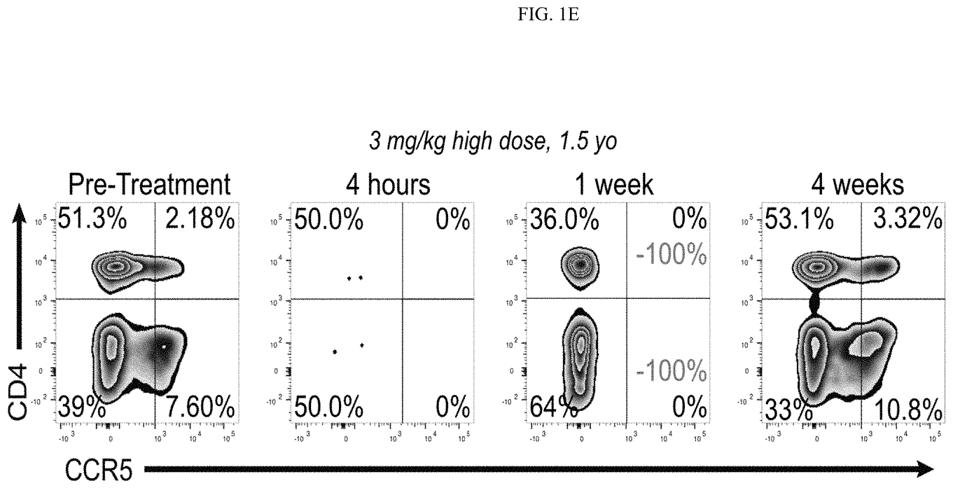

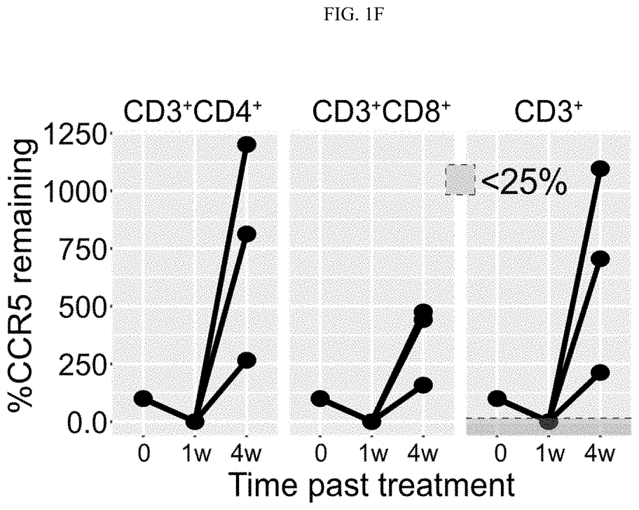

To assess the ability of the bispecific antibody to deplete CCR5⁺ cells in vivo, we initially administered 1 mg/kg antibody to two 1.5-year old rhesus macaques (FIGS. 1A-C). Most CD3⁺ cells were temporarily depleted from peripheral blood by 4 hours after treatment (FIG. 1A, second cytogram; note reduced ratio of CD8⁺ T cells to HLA-DR⁺ APCs) and reconstituted by one week. In addition, at four hours after treatment, a substantial reduction in the frequency of blood CCR5⁺ cells was identified in both CD4⁺ and CD8⁺ T cell subsets (65% and 69%, respectively; FIGS. 1B and 1C). By three weeks after treatment the CCR5⁺ cells had returned to their pre-treatment frequency (FIG. 1C).

We next tested the antibody at a higher dose, 3 mg/kg, in three 1.5-year-old macaques. At this dose the CD3⁺ lymphopenia was nearly complete by four hours after treatment (FIG. 1D). More significantly, however, we observed complete (100%) depletion of CCR5⁺ cells from both CD4⁺ and CD8⁺ T cell subsets by one week after treatment, in all animals treated (FIGS. 1E and 1F). Animals tolerated therapy well and continued to gain weight and maintain healthy blood counts up until necropsy. Finally, we treated two eight-month old macaques with 3 mg/kg of bispecific antibody. In these animals we also observed 100% depletion of CCR5⁺ cells from peripheral blood. These younger animals were necropsied 1 week after treatment to allow assessment of CCR5 depletion from tissues.

### Example 3. The CCR5xCD3 Bispecific Antibody Leads to Nearly Complete Depletion of CCR5⁺ Colonic and Lymph-Node T Cells In Vivo

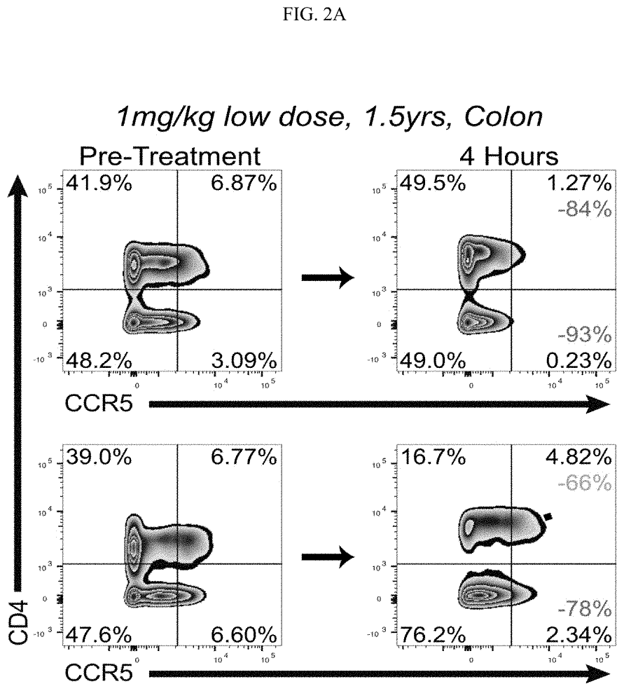

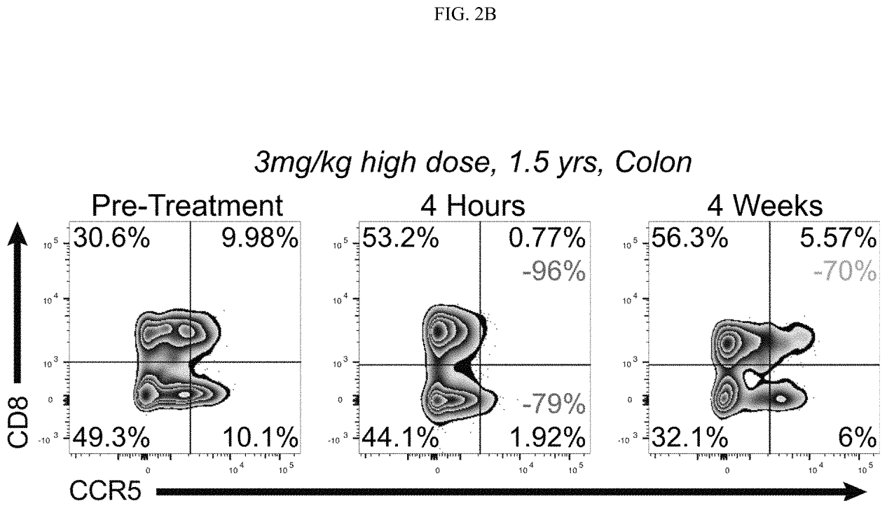

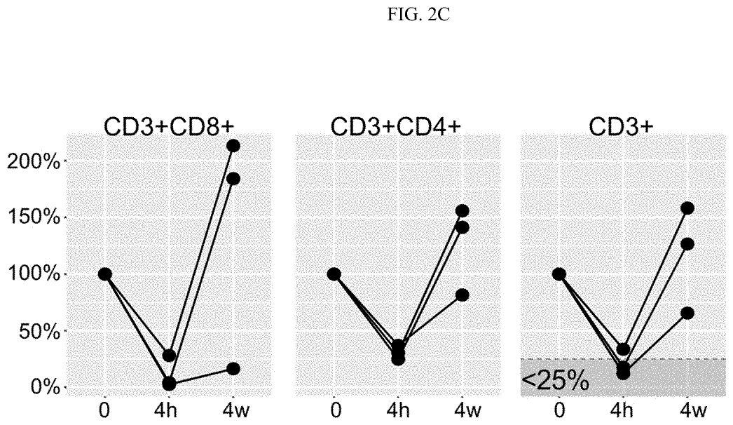

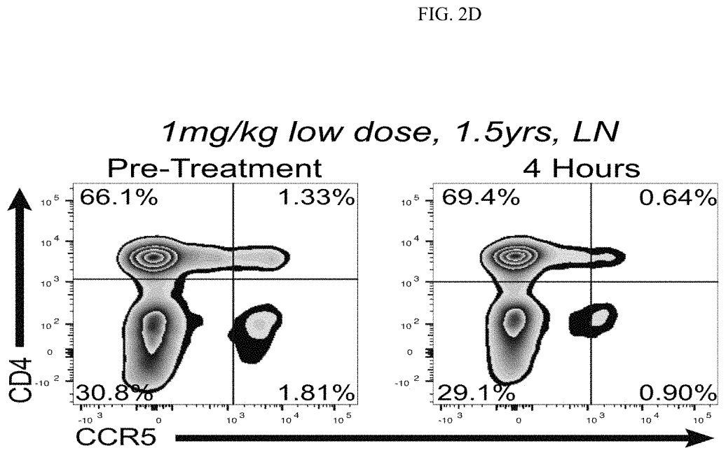

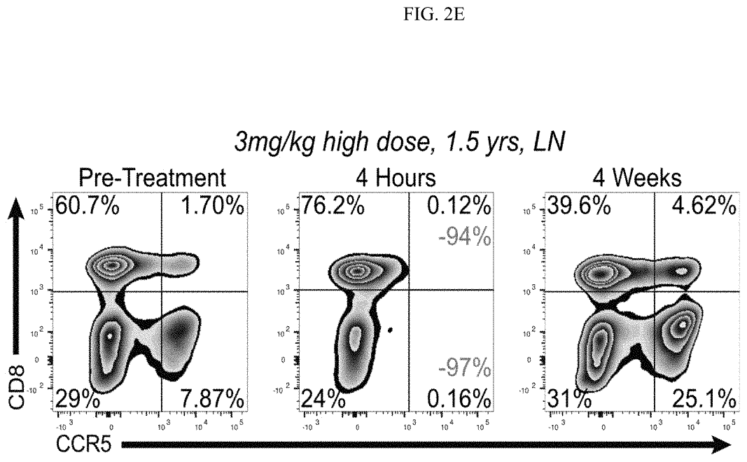

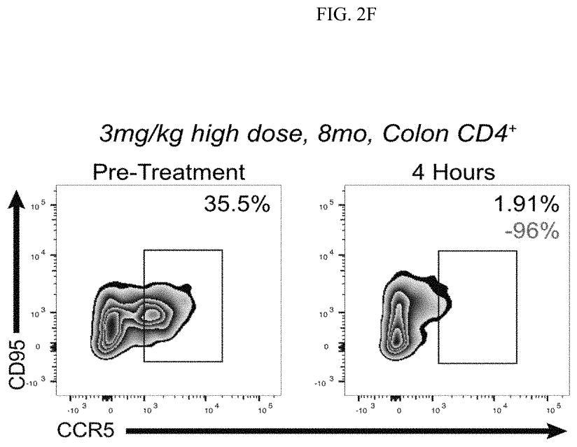

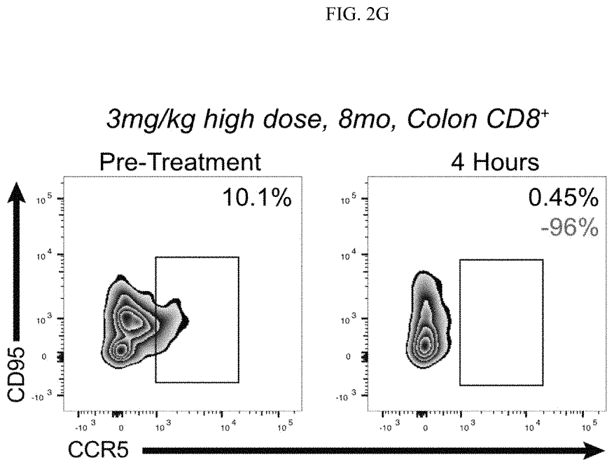

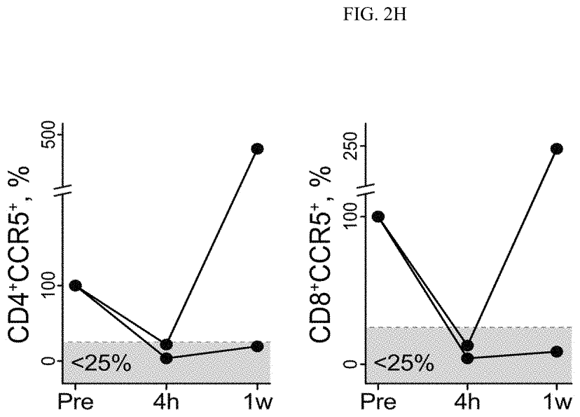

One of the greater challenges in drug delivery is to achieve therapeutic effect in tissues that may have poor drug penetration. For the CCR5xCD3 bispecific antibody, successful depletion of CCR5⁺ cells from intestine is of special importance because HIV undergoes robust replication and reservoir cells are known to exist in this anatomic site. Thus, to best promote cure in cooperation with host immune responses induced by therapeutic vaccination, depletion of CCR5⁺ reservoir cells from gut is presumably necessary. We assessed the frequency of CCR5⁺ cells among lamina propria lymphocytes (LPLs) from colon biopsies taken prior to treatment and four hours later from animals receiving 1 mg/kg or 3 mg/kg bispecific antibody. Each of the two 1.5-year-old macaques receiving 1 mg/kg bispecific antibody showed substantial depletion of CCR5-expressing CD8⁺ T cells from colon (78% or 93%; FIG. 2A). Only one animal treated with this low dose achieved substantial depletion of CD4⁺CCR5⁺ T cells, however (84%; FIG. 2A, top row).

In contrast, administration of high-dose antibody (3 mg/kg) to 1.5-year-old macaques resulted in ˜75% depletion of CD3⁺CCR5⁺ cells from the colon within four hours of treatment (FIGS. 2B and 2C). These animals were necropsied four weeks after treatment, at which time point one animal retained a depressed fraction of CCR5-expressing CD8⁺ T cells in gut, but the others saw the frequency of CCR5⁺ cells rebound to a level exceeding pre-treatment levels (FIG. 2C). Depletion of CCR5⁺ cells from lymph nodes followed a similar pattern, with some loss of such cells in animals receiving a low dose but complete depletion in those receiving a high dose of antibody (FIGS. 2D and 2E). Finally, we treated two 8-month-old macaques with 3 mg/kg bispecific antibody (high dose). These animals experienced uniform and profound depletion of CCR5⁺ T cells from colon tissue. We observed greater than 75% depletion of CCR5⁺ cells from both CD4⁺ and CD8⁺ T cell subsets in both animals (FIGS. 2F to 2H). We necropsied these animals one week after administering the antibody. Interestingly, one animal retained a substantial deficit of CCR5⁺ cells at 1 week, while the other demonstrated a significant rebound (FIG. 2H). In lymph nodes, these younger animals possessed a very low percentage of CCR5⁺ cells prior to treatment, but still saw substantial depletion of CCR5⁺ cells 4 hours after drug administration (FIG. 21).

### Example 4. Reservoir Depletion in Acute Infection Accelerates Decay of Viremia

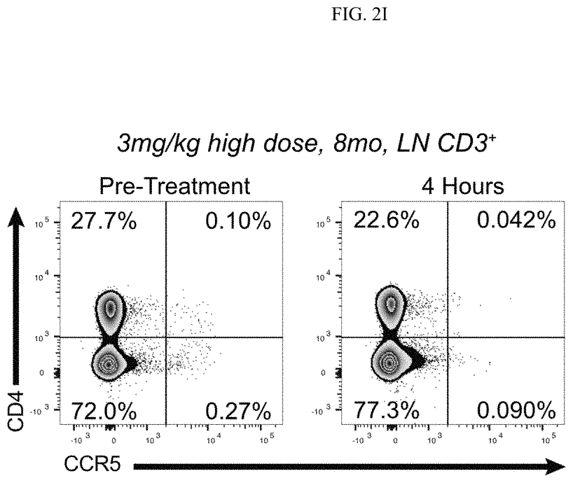

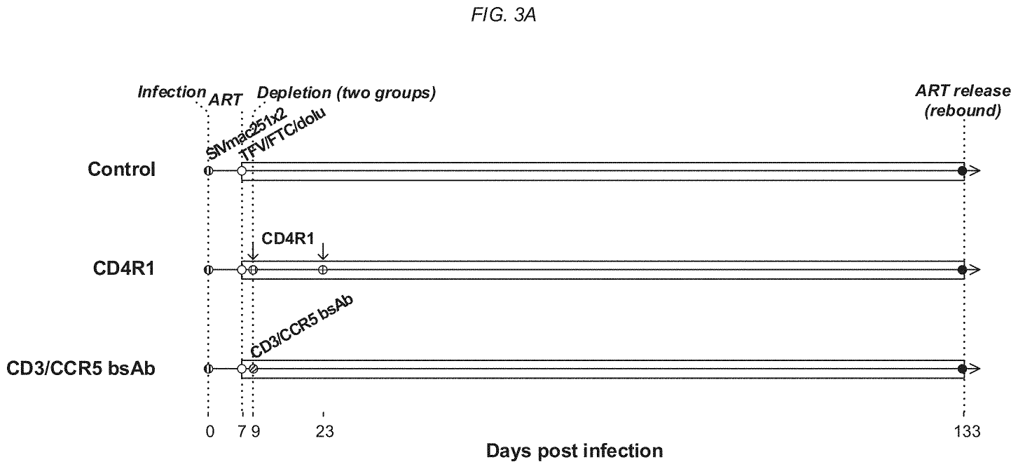

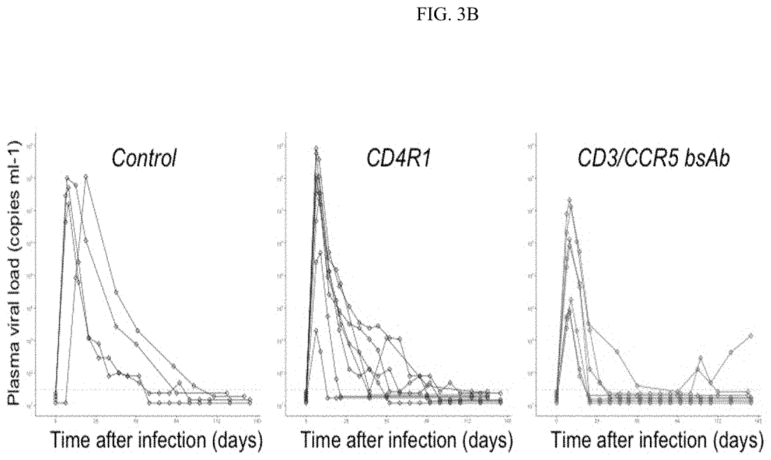

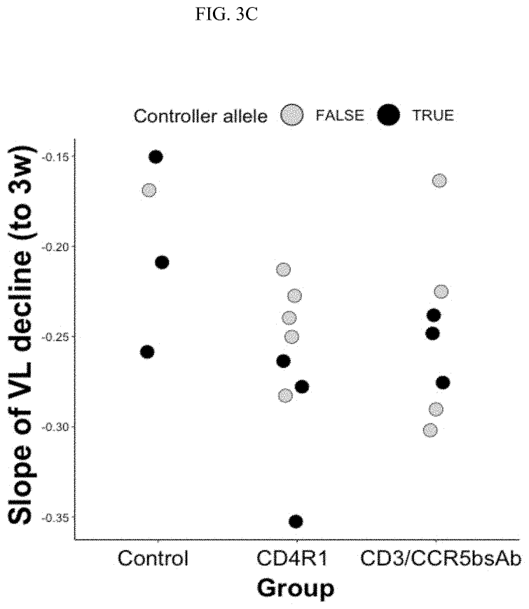

The infection and treatment protocol that was used to test efficacy modeled perinatal infection followed by early treatment, with administration of an experimental reservoir depleting agent soon after ART initiation (FIG. 3A). Neonatal macaques were infected at two weeks of age by inoculation with 50,000 TCID5o of SIVmac251 twice on the same day. Antiretroviral “triple therapy” (tenofovir, emtricitabine, and dolutegravir; ART) was started one week later to ensure peripheral dissemination of virus in all infected animals. Two days following ART initiation two groups were administered candidate reservoir-depleting therapies: group B received the depleting anti-CD4 antibody, CD4R1 (days 9 and 23 after infection), while group C received the anti-CD3/CCR5 bispecific antibody that we had shown depletes CCR5⁺ cells (day 9 after infection only). ART was withdrawn after 18 weeks and viral rebound subsequently monitored. 19/24 challenged infants were infected, with 3/5 uninfected animals having inherited a Mamu allele associated with SIV viral-load control (one A*01+/B*17+, one B*17+, one A*01+). Intriguingly, animals in groups B and C both experienced more rapid viral-load decay following reservoir depletion (FIGS. 3B and 3C; p=0.012 and 0.039, respectively), suggesting either a rapid effect of depletion or secondary effect on SIV spread, e.g., inhibition of viral entry. Mamu allele inheritance did not predict viral-load decay or time to achieve undetectable viral load; however, failure to inherit a controller allele was associated with viral “blips” after suppression was first achieved (3/9 animals with controller alleles and 9/10 without blipped; p=0.020 by Fisher's test).

### Example 5. Reservoir Depletion Achieves Functional Cure in Treated Infant Macaques

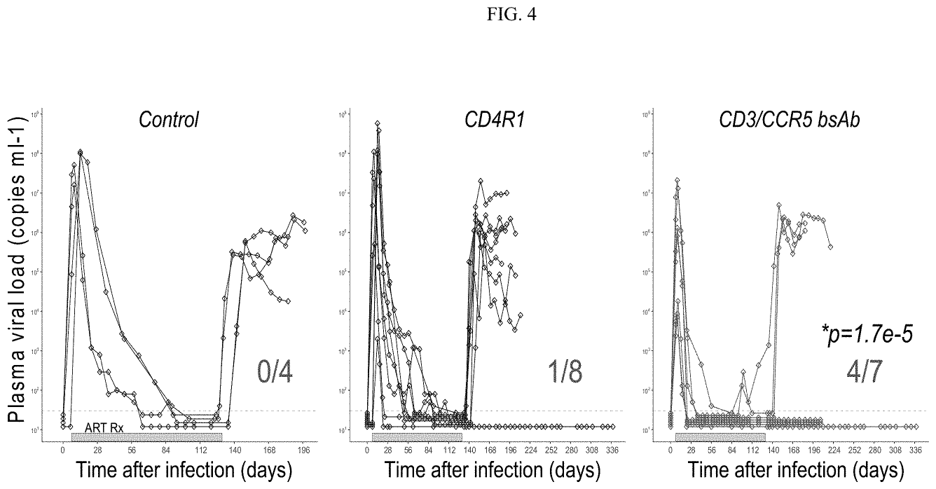

ART was withdrawn after 18 weeks of therapy to allow viral rebound. Peripheral viremia was rapidly observed in all control animals, 7/8 CD4R1-treated animals, and 3/7 bsAb-treated animals (FIG. 4). Four of seven bsAb-treated animals demonstrated stringent viral control that has persisted for the length of the experiment. Given an expected rate of cure of 2.7%, as preliminary data show for undepleted animals, the p-value for curing 4/7 animals was 1.5×10⁻⁵ (exact binomial test).

Controlling animals were exhaustively assessed for any signs of residual infection. Viral outgrowth assays found no residual replication-competent virus, despite detection of such virus in control animals with low-detectable viremia. Depletion of CD8+ cells from controlling animals did not allow observation of viral blips, despite effective depletion, suggesting that CD8⁺ T-cell responses were not the primary mechanism of control.

### Example 6. Optimize Reagents for CCR5 Depletion without T-Cell Activation

Immunotoxins and highly purified, optimized bispecific antibodies can achieve superior CCR5 depletion from intestine in the absence of immune activation. We previously tested two immunotoxins (RANTES fusion proteins) that had been described in the literature and demonstrated effective in vitro. In our hands, unfortunately, the proteins were effective only at high concentration in vitro and were ineffective in vivo. These sub-optimal results, considered in contrast to published work and typical results obtained clinically when employing toxins with low effective concentrations and high tissue penetration, suggested the likelihood that these unoptimized RANTES fusion proteins were poorly designed or misfolded in our expression system. The development path for CCR5 immunotoxins described below therefore includes iterative rounds of protein design, affinity testing, structure determination, and in vivo pharmacodynamic study.

In contrast, although CD3/CCR5 bispecific reagents had been described before, we followed a novel strategy based on the natural “arm exchange” observed with IgG4 molecules. The efficiency of this reaction allowed production of a bispecific molecule in the form of a true antibody—i.e., retaining its Fc effector region. The resulting molecule was at least 95% pure based on chromatographic analysis. Nonetheless, some amount of contamination with parental anti-CD3 antibody was thermodynamically inevitable; such contamination may cause the CD3⁺ lymphocyte margination and cytokine production resulting from administration of the first-generation bsAb. To eliminate this possibility, we now propose to eliminate contaminating anti-CD3 (lambda light chain). We also test other anti-CCR5 sequences and CCR5 arms for superior affinity to the target.

### Experimental Approach

To optimize reagents for CCR5 depletion without T-cell activation, we prepare and test in four parts new reagents that are free of contaminants and/or inherently not activating:
1a) Prepare bsAb free of parental anti-CD3 antibody by purification on a light chain-specific affinity column; 1b) Produce optimized immunotoxins based on CCR5 ligand-toxin fusions;
1c) Optimize bsAb for CCR5 affinity using new anti-CCR5 arms; and 1d) Test pharmacokinetics and pharmacodynamics of candidate agents in vivo, with a focus on CCR5 depletion from gut tissues without immune activation.

Each drug is be administered to three infant macaques (<6 months) in two doses separated by four weeks. Blood and tissue samples are be collected for at least one month after each administration. The primary outcomes of interest are CCR5 depletion, selectivity of depletion, markers of inflammation such as cytokine production, and anti-drug antibody production.

Purification of First-Generation bsAb Free of Contaminating Trace Anti-CD3

The production of bispecific molecules with two non-identical antigen-binding arms has the intrinsic purification challenge of removing “mispaired” (or residual) parental antibodies. To determine the extent of parental contamination, we regularly use Hydrophobic Interaction Chromatography (HIC) and ELISA assays to confirm asymmetric chain formation. While these methods are good to determine the conditions most favorable for bispecific manufacture, they are not sensitive enough to conclusively identify trace contamination with residual parental antibodies. Thus, we devised a new purification strategy to control parental contamination by leveraging the ability of affinity resins to selectively discriminate the different light chains. Under the new purification strategy, we use two sequential ‘polishing’ steps, to discard any symmetric byproduct. In the first, we use a Kappa-select column to retain molecules with kappa chains (present in the anti-CCR5 antibody) and discard the anti-CD3 parental molecule that only has lambda chains. We next use a Lambda-select column to discard any kappa chain-only parental. Albeit more complex, our new purification strategy greatly enriches for pure, asymmetric bsAb.

In-Vivo Testing

Blood volumes. The volume of blood drawn from macaques does not exceed 12 mL/kg/month, in accordance with primate-center guidelines. Given typical weights of infants, we draw 1.5 mL at each of time points 0 days, 5 days, 2 weeks, and 3 weeks post infection; 2.8 mL at time point 6 weeks; 4.5 ml at time point 8 weeks; and more than 5 mL at time point 12 weeks.

CCR5 depletion assessment. CCR5 depletion is assessed by staining of cell samples (e.g., from blood, colon, lymph node, or other tissues) with fluorescently labeled antibodies to CCR5 and to other molecules defining T-cell subsets (CD3, CD4, CD8, CD95, CCR7, etc.). The stained samples are washed and the presence of CCR5-expressing cells assessed by flow cytometry. Alternatively, the presence of cells expressing CCR5 mRNA in tissue samples can be assessed by RT-PCR, a nucleic-acid amplification technique.

Anti-drug antibodies (ADAs). ADAs to immunotoxins are assessed by direct ELISA using an anti-rhesus macaque IgG detection reagent. Testing ADAs to administred mono- or bispecific antibodies is complicated by the need for a detection reagent that is reactive to the ADAs but not to the drug (antibody) coating the plate. Therefore, the detection reagent in this case is anti-kappa or anti-lambda light chain, whichever is not reactive to the drug. In the case of bispecific antibodies, plates are coated with one parental antibody at a time.

Necropsy. A large number of samples are collected, including those from key anatomic reservoirs such as spleen, lymph node, thymus, lumbar spinal cord, GI tract, and lung. Fluorescence-activated cell sorting is used to fractionate the CD4⁺ T cell population. Aliquots of cells are stained with a panel including antibodies specific for CCR5, CD3, CD4, CD8, CD14, CD25, CD69, CD95, CD28, and HLA-DR.

### Interpretation of Data

The hypothesis of this experiment is that superior CCR5 depletion can be achieved without immune activation, i.e., that the activation seen after administration of the first-generation bsAb was not important for depletion. Key outcomes therefore include the extent and specificity of CCR5 depletion from intestine as well as the phenotypic and circulating measures of immune activation.

### Statistical Analysis

Importantly, given the potency of the agent we already have in hand (75-100% depletion from intestine), we seek agents that are nearly 100% effective in depleting CCR5⁺ cells from blood and tissues. Only those agents are realistic possible replacements for bsAb in our SIV-cure studies. Thus, the primary analysis is a comparison of treated animals to untreated controls, in which we simply seek treatments that are close to 100% effective. In secondary analyses, generalized linear mixed models (GLMMs) can be used as the over-arching analysis framework for detecting differences, e.g., in the extent of CCR5 depletion or in cytokine production. Use of mixed models facilitates accommodation of the within-animal dependence induced by serial measurements, along with adjustment for key covariates. Software (R, SAS) for performing such analyses is well established, as are graphical and other diagnostic approaches for exploratory modeling and model checking. Taking the level of circulating IL-6 as an example, using three animals per group can give us 80% power to detect a rise from the background level observed in preliminary experiments of 3.4±1.3 μg/mL to 7.4 μg/mL. Far greater rises in circulating IL-6 are observed in macaques experiencing widespread T-cell activation.

### Alternative Approaches

The overriding goal of work in this example is to generate candidate CCR5-depletion reagents that are non activating (or less activating than the bsAb reagent used previously) and can be tested for curative efficacy in Specific Aims 2 and 3. bsAb free of parental anti-CD3 can be produced by any of several alternative selection strategies, including adding a cleavable tag to the CCR5 light chain or using a protein-L column for kappa light-chain selection. Immunotoxins can be produced using any of two known CCR5 ligands and ˜10 widely known toxins, connected by a variable-length spacer that can influence toxin internalization and cell killing. The number of possible optimized bsAbs with increased CCR5 affinity is large and constrained only by the CCR5 clones available. We have access to a substantial number of clones that are widely known and whose sequence can be obtained by hybridoma sequencing. Other alternatives include, e.g., true BiTEs lacking an Fc region, antibody-based immunotoxins, and even generation of new anti-CCR5 clones.

### Example 7. Test if T-Cell Activation Contributes to SIV Cure Achieved in Newborn Macaques

It is hypothesized that SIV cure achieved by CD3/CCR5 is due primarily to reservoir depletion, without an important contribution from bsAb-induced T-cell activation. Several aspects of our results suggest that we can achieve an equivalent cure rate in absence of T-cell activation. First, during the previous experiments, we evaluated the potential for cure using either bsAb or CD4R1—an anti-CD4 antibody that causes CD4 depletion with minimal or no T-cell activation. Although bsAb was more frequently effective, CD4 depletion did cure a single recipient animal (of eight treated), suggesting at a minimum that T-cell activation is not necessary for cure. Second, our studies in the previous experiments were conducted in newborn macaques that were both prone to poor T-cell responses to SIV and in fact demonstrated negligible adaptive immune responses post cure. Animals achieving cure manifested lower adaptive immune responses than animals that were not cured. Finally, despite considerable heterogeneity in the extent of depletion, we noted an association between depletion and cure, suggesting that depletion is an important driving force.

### Experimental Approach

24 newborn monkeys are infected with SIVmac251 on day 14 of life, then receive ART treatment and reservoir depletion exactly as in the previous experiments. The agents used for depletion can depend on the results of the previous example; however, we plan to include (i) purified first-generation bsAb lacking parental anti-CD3 (n=8); (ii) one immunotoxin (n=8); and (iii) one purified second-generation bsAb with superior CCR5 binding affinity (n=8). Immune responses and occurrence of cure are followed in all groups. The results are compared with those seen after no treatment or administration of unpurified first-generation bsAb. Data for these latter control groups is from Preliminary Data (n=4 and n=7, respectively) and ongoing studies (n=4 additional animals each) to provide a total of 8 untreated and 11 bsAb-treated animals for comparison.

Newborn Macaque Screening and Assignment

Cord blood samples are collected from newborn macaques at birth and tested by PCR for Mamu alleles A*01, B*08, and B*17, all associated with relative SIV control. The newborns are then assigned to the three arms of this study so as to balance the distribution of animals with controller alleles to the extent possible.

Infection and Treatment

Newborn macaques are infected on day 14 of life by an established oral inoculation regimen (two doses of 100,000 TCID₅₀ of SIVmac251 on the same day; see ref 16). All begin ART treatment seven days after infection. Treatment consists of “triple therapy” with tenofovir, 20 mg/kg s.c. qd; emtricitabine, 50 mg/kg s.c. qd; and dolutegravir, 3.25 mg/kg s.c. qd. Reservoir-depleting bispecific antibodies or immunotoxins are first administered nine days after infection. Blood samples are taken on the day of infection (i.e., 14 days after birth), 5 days after infection, and on post-infection weeks 1, 2, 3, 6, 8, 12, 16, 20, 24, 28, and 32, and at least every four weeks thereafter. Lymph node biopsies are taken one and two months after treatment initiation. ART is discontinued 19 weeks after infection to allow viral rebound. The animals are necropsied after rebound viral loads have stabilized, but no sooner than 25 weeks after infection. Controlling animals are maintained for a longer period to allow detailed testing for residual virus.

Blood Volumes

The volume of blood drawn from macaques does not exceed 12 mL/kg/month, in accordance with primate-center guidelines. In the case of infants, given their usual weights, we draw 1.5 mL at each of time points 0 days, 5 days, 1 week, 2 weeks, and 3 weeks post infection; 2.8 ml at 6 weeks; 4.5 mL at 8 weeks; and more than 5 mL beyond 12 weeks. These volumes are more than sufficient for viral-load testing and periodic immune-response assays, in additional to viral outgrowth assays after rebound (beyond 18 weeks).

Viral Nucleic Acid Analysis in Plasma and PBMC

vRNA and vDNA are evaluated in plasma and cell samples initially using quantitative RT-PCR. To accommodate the low sample volumes available from infant macaques, the extracted RNA is run in 12 replicates in a 384-well format. The advantage of this format is that reliability of positive determinations is enhanced and the threshold limit much lower than standard. Using this method, the lower limit of detection in a 100 microliter sample is 16 copies/mL.

All samples that are initially found to be negative are then be tested in an ultrasensitive assay. This approach uses a hybrid real-time and digital PCR technique to allow input of a large amount of test sample in the first round of testing, critical for detection of rare sequences in a large specimen amount. An extremely broad dynamic range of determinations is obviously possible, but most significant is the ability to quantify very low target levels, on the order of 1 copy per number of test aliquots (i.e., one copy per 10′ to 10⁸ cells), with good reliability.

CD4⁺T Cell Isolation at Post-Infection Week 6 and Beyond

At later time points, when enough cells are available for isolation, viral nucleic acid levels are be assessed in the circulating CD4⁺ population. CD4⁺ T cells are isolated using paramagnetic beads and tested using the amplification techniques described above.

Inducible Virus in CD4⁺ T Cell Reservoirs at Week 12 and Beyond

Aliquots of PBMC or isolated CD4⁺ T cells isolated are tested for the presence of inducible SIV replication. We anticipate availability of at least 250,000 such cells for stimulation and culture in vitro at the 12th week of infection and beyond. Each cell population is co-cultured with CEMx174 cells and monitored by p27 antigen capture ELISA according to established methods.

Evaluation of T Cell Activation at Week 6 and Beyond

Activated T cells present at each time point are assessed in 30 microliters whole blood (to conserve sample) using a flow cytometry panel including HLA-DR, CD38, and Ki-67.

Immunologic Analysis of Infected Animals at Week 6 and Beyond

The quantity and quality of virus-specific T cell responses are assessed by cytokine flow cytometry (CFC) analysis of whole blood samples. Assay wells containing 50 microliters whole blood are stimulated with vehicle (negative control for DMSO toxicity), overlapping SIV p27 gag peptides (AIDS Research and Reference Reagent Program), or PMA/ionomycin (positive control). All wells receive anti-CD28 and anti-CD49d at a concentration of 2 μg/mL. GolgiPlug (BD Biosciences) is added one hour after the start of incubation. Five hours later, samples are harvested by centrifugation, fixed, permeabilized, and stained using fixable live-dead stain as well as antibodies reactive to CD3, CD4, CD8, CD27, CD45RA, IL-2, IL-17, IFN-γ, and TNF-α. The fraction of cytokine-secreting CD4⁺ and CD8⁺ T lymphocytes is determined by flow cytometry using a BD LSR-II.

Detection of Sly-Specific Antibody Responses

SIV-specific immunoglobulin G (IgG) in plasma samples is detected by ELISA as previously described. Depending on the outcome, ACDVI and neutralizing antibody assays can also be performed.

### Interpretation of Data

The data needed to address the hypothesis that SIV cure is attributable primarily to reservoir depletion and not to the immune activation include viral loads and cell-associated viral DNA data (indicating if cure has been achieved or, alternatively, showing the time and rate of viral rebound); data relevant to the extent of CCR5 depletion from blood and tissues; and indications of immune activation, most importantly including cytokine production after depletion. The hypothesis predicts that optimized depleting agents without activating properties can achieve cure at a rate that is at least non-inferior to that achieved with first-generation bsAb (57%).

In addition, however, there are a variety of ancillary questions that can be answered by our data. For example, we want to know if animals that are cured within a group show lesser presence of the virus in reservoirs at the time of ART withdrawal than those not cured, lending further support to depletion as the mechanism of efficacy. This question can be answered by examining DNA viral loads in isolated CD4⁺ T cells. Further, whether T cell activation, virus-specific T cell responses, and/or T-reg function are related to occurrence of cure can also be investigated. These measures of T cell function exhibit substantial inter-individual variability, particularly among infant animals, and it is be important to determine if they have a strong association with viral reservoir levels and eventual cure. We may find, for example, that infants with poor CD8⁺ T-cell responses or robust Treg development permit greater establishment of SIV in reservoirs. Such a finding would implicate T cell responses in the mechanism of cure and suggest an explanation for inconsistent outcomes that may be achieved after early intensive ART.

### Statistical Analysis

Fisher's exact test is used to statistically compare proportions of macaques that achieve functional cure (as defined above) in each of the two groups. Generalized linear mixed models (GLMMs) can be used as the over-arching analysis framework for detecting differences in SIV reservoirs, T cell activation, and other longitudinal outcomes. Groups of 8 animals provide more than 90% power for detection of a 5-day difference in time to viral rebound, if the standard deviation of these values is —2.75 days, as we observed in our prior experiments.

### Alternative Approaches

The finding that CD3⁺ lymphopenia and/or inflammation are essential to cure would be tremendously important in the field. CD3⁺ lymphopenia would suggest immunosuppression or lymphodepletion are crucial contributors via mechanisms as yet unknown.

### Example 8. Determine if Recipient Age or Reservoir Maturity Limit SIV Cure by CCR5 Reservoir Depletion

It is hypothesized that, like newborns, juvenile macaques treated early can be cured by CCR5 depletion, but delay of treatment to 14 days after infection limits the frequency of sterilizing cure. Rationale: The assumption in the field is that transmitted/founder viruses are CCR5 tropic, regardless of the age of infection. Thus, CCR depletion can be equally effective in older vs. younger animals. We chose initially to study newborn infants only because in this population the precise time of HIV infection is more often known (i.e., at birth) and because treatment can more often be initiated very early. The impact of delayed treatment on chance of cure is perhaps harder to predict. Studies of post-exposure prophylaxis and rebound make clear that the reservoir is dynamic in early infection, with later treatment leading to a larger reservoir, which in turn requires only a short time to generate recrudescent viremia after ART withdrawal. In addition, because CCR5 is an activation marker, it seems possible that infected CCR5⁺ cells can lose expression, leading to accumulation of reservoir cells that are invulnerable to bsAb. On the other hand , a longer period before reservoir depletion allows more time for generation of a functional host immune response, which likely contributes to post-treatment control. Indeed, the potency of this combination of reservoir depletion and host immune responses (e.g., stimulated by a vaccine) is the basis of this invention. Because in this example no vaccine will be given, we believe that later treatment could lead to reduced frequency of sterilizing cure, though possibly “functional” cures with residual reservoir cells will be more common.

### Experimental Approach

A CCR5 depletion strategy is selected from those tested in the previous example using the criteria of (i) frequent cure, (ii) minimal immune activation, and (iii) efficient CCR5 depletion from tissues, in that priority order. This strategy is tested in newborns treated at seven days in the previous example; here we assess efficacy in older animals and in the context of a later ART start. Similarly to the previous example, three groups of rhesus macaques are infected by high-dose oral inoculation (two doses of 50,000 TCID50 of SIVmac251 on the same day) and treated with ART beginning 7 or 14 days after infection. The experimental groups are (A) newborn macaques treated with ART starting at 14 days post infection, (B) juvenile macaques (1-3 years old) treated with ART starting at 7 days, and (C) juvenile macaques treated with ART starting at 14 days. ART will be discontinued after 18 weeks (or longer if suggested by the results of Example 6) and parameters including viral load, immune response to virus, and presence of the virus in reservoirs are assessed over time exactly as described in the previous example.

### Interpretation of Data

Data gathered here are analyzed together with those from the appropriate control group in the previous example to determine if similar frequency of either sterilizing or “functional” cure by CCR5 depletion is achieved. Alternatively, even if this treatment does not achieve functional cure in older or later-treated animals, we may observe evidence of increased control over virus (e.g., lower plasma vRNA levels) after drug withdrawal. We can track both the absolute level of cell-associated SIV DNA (in copies per cellular genome) and the percentage subset contribution to the total resting SIV reservoir, obtained by combining absolute levels with the relative frequency of each subset in blood. In the short term, we expect that CCR5 depletion under cover of ART to reduce the absolute levels of SIV nucleic acid detected in all subsets, as cells containing SIV genomes are depleted and eventually replaced. Nevertheless, it is possible that the increased homeostatic proliferation associated with CD4⁺ T cell depletion can lead to an increase in the level of SIV DNA found in CD4⁺ T cells, either before or after discontinuation of ART.

Later CCR5 depletion could be associated with host immune responses to viral proteins that contribute to “functional cure”, i.e., elite control over viral replication despite demonstrable continued presence of the virus.

It is possible that CCR5 expression is driven in newborn animals by cellular differentiation (conferring a stable phenotype) but in older animals by T-cell activation (which might be expected to cycle more rapidly and result in conversion of CCR5⁺ reservoir cells to CCR5-negative). Although it might seem that later ART treatment is an insurmountable barrier, we have in fact observed that animals treated as late as four weeks after infection, allowed to rebound, can control viremia to ˜10⁴ copies/ml, a level below what would have been expected in absence of ART. We consider it possible that CCR5 depletion could convert some such situations into functional cures, most likely by removing a portion of the viral reservoir and allowing the host immune system to maintain its efficacy. Therapeutic vaccination will further augment the chance that incomplete reservoir depletion is converted to HIV cure.

### Statistical Analysis

The statistical techniques to be used here are the same as those described in the previous example. Fisher's exact test can be used to statistically compare proportions of macaques that achieve functional cure in each of the four groups (three groups from this example and the relevant control group from the previous example). Generalized linear mixed models (GLMMs) can be used as the over-arching analysis framework for detecting differences in SIV reservoirs, T cell activation, and other longitudinal outcomes. Survival analysis can be used for time-to-event data, e.g., time to viral rebound. Groups of 10 animals provide more than 90% power for detection of a 5-day difference in time to viral rebound, if the standard deviation of these values is ˜2.75 days, as observed in our prior experiments.

### Alternative Approaches

Poor depletion can be due to a different pattern of CCR5 expression. Another possibility, however, might be different developmental origins of CCR5 expression in juvenile macaques. Such a possibility could be further investigated by performing BrDU labeling experiments in juvenile monkeys to determine the half life of CCR5⁺ cells in circulation. One can imagine that the half life in newborns could be long due to stable expression of the subset on a developmentally defined population—in the setting of low exogenous T-cell activation—but that the half-life could be short in juvevniles due to cycling of cells from an inactivated to activated state.

### Example 9. Depletion of Reservoir Cells and Subsequent Vaccination

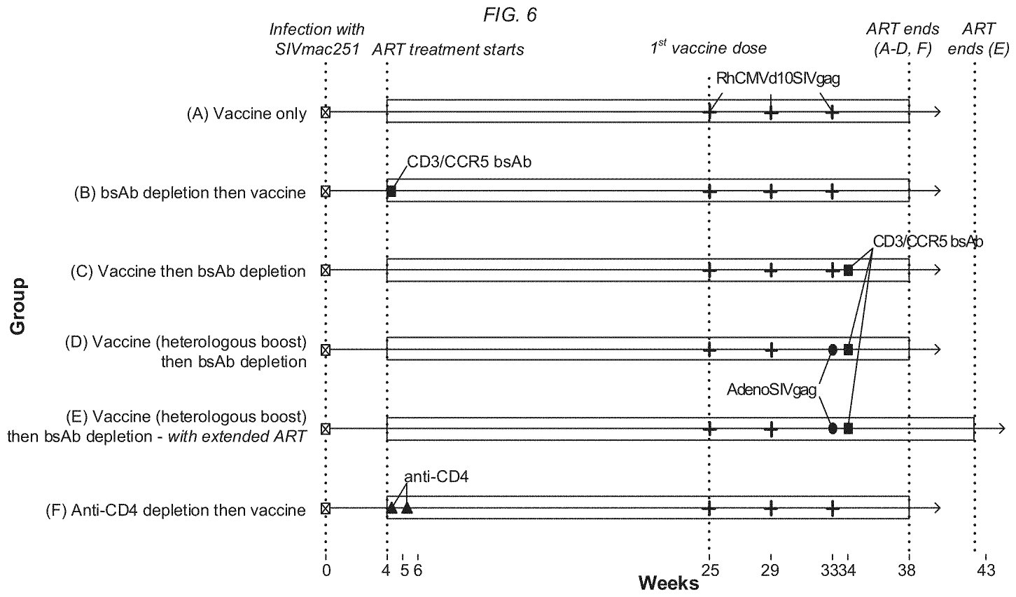

Here we provide therapy for SIV-infected macaques on antiretroviral therapy that consists of reservoir depletion two days after ART initiation, followed by a series of vaccinations several months later. The combined actions of ART and anti-CD3/CCR5 bispecific antibody treatment reduce the number of reservoir cells harboring SIV to a lower level than is achievable with ART alone, which allows the immune responses elicited by vaccination to effectively control the few remaining cells. The result is that bsAb-treated animals exhibit superior control after ART removal, as reflected in lower viral loads in blood and/or tissues. FIG. 6 shows exemplary timelines (groups B and F).

### Experimental Approach

We assess the SIV reservoir and the extent of control after ART withdrawal in two groups of ART-suppressed rhesus macaques receiving (A) vaccine only or (B) combination therapy consisting of anti-CD3/CCR5 bispecific antibody treatment two days after ART start, followed by RhCMVdeltaIL-10-vectored vaccinations at weeks 25, 29, and 33 (FIG. 6; groups A and B). T cell chemokine receptor expression, T cell localization, T cell in vitro function, and viral reservoirs are then followed in parallel throughout 13 weeks after priming vaccination (while maintaining ART) and a subsequent ˜10 weeks after ART withdrawal. Macaques receiving combination therapy (group B) manifest a reduced viral reservoir and superior control after ART withdrawal, as evident in lower viral loads or complete absence of virus.

### Rhesus Macaques and SIVmac251 Infection

The specific pathogen free (SPF) resource was established in order to provide animals free of retroviral agents (SIV, simian T-lymphotropic virus, and Type D simian retrovirus) that might confound HIV/SIV-related research. When it became clear that other agents, particularly RhCMV, must also impact upon on disease pathogenesis and vaccine responses, the SPF Level 2 colony was created to provide animals free of RhCMV, simian foamy virus, and rhesus rhadinovirus. SPF Level 2 animals of both sexes are used for all work in this application. Juveniles (2-3 years old) are used to minimize the weight of antiretroviral drugs required. All animals are infected by non-traumatic intrarectal inoculation of 10⁴ IU of SIVmac251.

Antiretroviral therapy is initiated4 weeks post infection and consist of co-formulated “triple therapy” with 5.1 mg/kg tenofovir disoproxil fumarate, 40 mg/kg emtricitabine, and 2.5 mg/kg dolutegravir subcutaneously each day. The animals are treated for 21 weeks before vaccination and 34-38 weeks before ART withdrawal. We have previously demonstrated complete suppression with this ART regime (to <30 copies/ml) after 20 weeks of therapy in most macaques treated.

Anti-CD3/CCR5 Bispecific Antibody Treatment for Depletion of SIV-Reservoir Cells

An antibody capable of engaging both CD3 and CCR5 was engineered using the controlled Fab arm exchange method. Hybridomas expressing anti-CCR5 or anti-CD3 antibodies were obtained. The heavy and light chain variable regions of both antibodies were sequenced and CDR5 grafted into rhesus macaque frameworks. Both antibody heavy chains were constructed with rhesus macaque IgG1 constant regions containing mutations in CH3 to enable Fab arm exchange.

Both parental antibodies' engineered heavy and light chain genes were inserted into an expression vector and used for large-scale transient transfection of CHO cells through the ExpiCHO expression system (Life Technologies). Antibodies expressed after culture in serum-free medium were purified using protein A affinity chromatography and adjusted to 3.5 mg/ml in PBS pH 7.0. Equimolar quantities of both antibodies were mixed and incubated at 37° C. for 90 min in the presence of the mild reducing agent, 2-mercaptoethylamine. After this incubation step, the reductant was removed by extensive dialysis against PBS pH 7.0.

Efficiency of Fab arm exchange was confirmed by hydrophobic interaction chromatography (HIC) and capillary isoelectric focusing (cIEF)—analytic methods which could resolve the parental and bispecific antibodies. Briefly, for HIC, samples of individual parental antibody, a mixture of two parental antibodies, or bispecific product were loaded onto a 7.8 75 mm ProPac HIC-10 column (Thermo Fisher Scientific) by a predilution to 1.0 mg/ml in HIC equilibration buffer (100 mM phosphate containing 1.2 M ammonium sulfate, pH 7.0). The high ionic strength in the loading buffer facilitates the interaction between the hydrophobic chromatographic medium and the hydrophobic patches present on protein molecules. In the later separation, the salt concentration is gradually decreased in order of increasing hydrophobicity, and the antibodies are eluted off of the HIC column. cIEF is a method that separates native protein species by their isoelectric points (pI). The antibodies were characterized under their condition using a PA 800 Protein Characterization System (Beckman Coulter, Inc., Fullerton, CA) equipped with UV detector. 30.2 cm long, 50 lm I.D. neutral capillary, maintained at 20° C., was used for separation. Test samples were injected after dilution to 0.2 mg/ml in a diluent containing four synthetic peptide pI standards, Pharmalyte 3-10 carrier ampholytes, and a gel matrix. By subsequently changing the pH of the mobile phase from basic to acidic, species with different charges are focused with different migration times. The pI of each peak is determined by a linear calibration curve calibrated by the internal pI standards.

The finished anti-CD3/CCR5 is administered intravenously at a dose of 3 mg/kg.

Viral Nucleic Acids

Plasma viral loads are followed by quantitative RT-PCR. Cell-associated viral RNA and viral DNA (i.e., viral nucleic acid in reservoir cells) are measured when viral loads under ART treatment fall below 200 copies/ml and continuing to the end of the experiment.

RhCMVd10SIVgag Vaccine Administration

RhCMVd10SIVgag vaccine is administered subcutaneously at a dose of 5×10⁶ plaque forming units per animal. Three immunizations are given with four weeks between each administration (see FIG. 6; weeks 25, 29, and 33).

Therapy is withdrawn 13 weeks after priming vaccination and the animals followed for at least 10 subsequent weeks.

Blood Samples

The volume of blood drawn does not exceed 12 ml/kg/month, in accordance with primate-center guidelines. Given typical weights of juveniles, we are able to draw up to 48 ml each month. These volumes are more than sufficient to perform the assays we envision.

Biopsies and bronchoalveolar lavage (BAL) are performed by very experienced primate-center staff.

Host Immune Phenotypes

Host immune phenotypes, including T cell subpopulations, activation, exhaustion, and homing markers (e.g., CXCR5 and CCR7) are followed by flow cytometry using standard techniques. For assessment of antigen-specific T cell responses, assay wells containing up to 1M PBMC, LNMC, or BAL cells are stimulated with vehicle (negative control for DMSO toxicity), overlapping SIV peptides, specific peptides with known Mamu-E restriction (e.g., Gag(69) as described above), or PMA/ionomycin (positive control). All wells also receive anti-CD28 and anti-CD49d at a concentration of 2 μg/ml. Inhibitors such as VL9 peptide or anti-HLA-antibodies are applied one hour before stimulation begins. GolgiPlug (BD Biosciences) are added one hour after the start of incubation. Five hours later, samples are harvested by centrifugation, fixed, permeabilized, and stained using fixable live-dead stain as well as antibodies reactive to CD3, CD4, CD8, CD27, CD45RA, IL-2, IL-17, IFN-γ, and TNF-α. The fraction of cytokine-secreting CD4⁺ and CD8⁺ T cells are determined by flow cytometry on a Fortessa.

T Cell Localization

T cell localization by histologic analysis is performed with previously described methods. To test if RhCMVd10-induced T cell infiltration to B cell follicles includes Gag-specific T cells, Mamu-E tetramers are used for immunofluorescent staining. Tetramers are obtained with both relevant (known Mamu-E restricted) and irrelevant control peptides to facilitate interpretation of the data.

Viral Inhibition Assays

Viral inhibition assays are performed to determine if CD8⁺ T cells from the treated animals have SIV inhibitory activity in vitro. Viral replication inhibition assays have become a standard tool in human vaccine trials to provide a useful functional readout. Briefly, autologous CD4⁺ target cells are stimulated with PHA for 3 days and infected with SIVmac251 challenge stock at several MOIs. Targets are then cultured alone or in the presence of CD8⁺ effectors; after 6 days, cells are stained for intracellular p27. Expression levels of class I molecules (antibody clone W6/32) and Mamu-E molecules on targets are measured throughout.

Evaluation of the SIV Reservoir at Necropsy

A large number of samples are collected, including those from key anatomic reservoirs such as spleen, lymph node, thymus, lumbar spinal cord, GI tract, and lung. Fluorescence-activated cell sorting is used to fractionate the CD4⁺ T cell population. Aliquots of cells are stained with a panel including antibodies specific for CCR7, CD3, CD4, CD8, CD14, CD27, CD95, CD28, CD200, CXCR5, HLA-DR, and PD-1. T follicular helper cells (Tfh; CD3⁺CD4⁺CD95⁺CD28⁺CXCR5⁺PD⁻1⁺), central memory CD4⁺ T cells (TCM; CD95⁺CD28⁺CXCR5⁻), effector memory CD4⁺ T cells (TEM; CD95⁺CD28⁻), and CD4⁺ monocytes/macrophages (CD3⁻CD14⁺CD4low) are sorted into individual tubes on a BD FACS Aria sorter. Samples from these reservoirs are assessed for viral cell-associated RNA and DNA. Tissues are preserved in formalin and OCT for immunohistochemistry and immunofluorescence to assess CD8⁺ T cell localization.

Co-Culture Assay for Replication-Competent Virus within Sorted CD4⁺ T Cells, Including Tfh

After necropsy, graded numbers of sorted CD4⁺ memory T cell subsets or Tfh (10³, 10⁴, or 10⁵ cells) are tested for presence of replication-competent virus by cultivation of these sorted cells with 10⁵ CEMx174 cells in 24-well plates, followed by flow cytometric analysis of intracellular SIV-Gag p27 expression. Cocultured cells are harvested and analyzed at days 13-36, with quantitative comparisons of the extent of infection in cultures containing different CD4⁺ memory T cell populations performed at the earliest time point of maximum expression of Gag p27 in the cultures of any of the CD4⁺ T cell subsets tested. The important result of this assay is an assessment of how much replication-competent virus is found in potential reservoir cells in groups A-B. The reservoir depletion performed in group B results in a smaller reservoir, with less recovery or replication-competent virus.

CCR5 Depletion Assessment

CCR5 depletion is assessed by staining of cell samples (e.g., from blood, colon, lymph node, or other tissues) with fluorescently labeled antibodies to CCR5 and to other molecules defining T-cell subsets (CD3, CD4, CD8, CD95, CCR7, etc.). The stained samples are washed and the presence of CCR5-expressing cells assessed by flow cytometry. Alternatively, the presence of cells expressing CCR5 mRNA in tissue samples can be assessed by RT-PCR, a nucleic-acid amplification technique.

Anti-Drug Antibodies

Antibodies against the idiotypes of the bsAb are tested by standard ELISA, using the parental antibodies to coat the plates. By using a detection antibody that is reactive to the light-chain type not found in the parent, antibodies generated in macaques receiving combination therapy can be assessed.

Viral Nucleic Acid Analysis in Plasma and PBMC

Plasma viral RNA is evaluated initially using quantitative RT-PCR. The extracted RNA is run in 12 replicates in a 384-well format, which enhances the reliability of positive determinations and lowers the detection threshold. The lower limit of detection in a 100 microliter sample is 16 copies/ml. Rebound is defined in this study as two consecutive SIV RNA levels ≥200 copies/ml.

All samples that are initially found to be negative will then be tested in an ultrasensitive assay. This approach uses a hybrid real-time and digital PCR technique to allow input of a large amount of test sample in the first round of testing, critical for detection of rare sequences in a large specimen amount. Most significant is the ability to quantify very low target levels, on the order of 1 copy per number of test aliquots (i.e. one copy per 10⁷ to 10⁸ cells), with good reliability.

### Interpretation of Data

Due to vaccination, animals in groups A-B exhibit T-cell responses against the SIV gag gene that are superior to those seen in unvaccinated controls. The animals also demonstrate a greater frequency of CD8⁺ T cells within B cell follicles. In addition, most importantly, the animals in group B demonstrate lower frequencies of infected Tfh and/or lymph node-resident T cells (reservoir cells), as seen in CEMx174 co-culture assays.

We also assess the functional (killing) capacity of T cells from each vaccinated animal, demonstrating that our vaccine regimen elicits functional cells capable of cooperating with anti-CD3/CCR5 bsAb to eliminate reservoir cells over time, presumably as the viral genomes within them periodically re-activate, rendering the cells vulnerable to CD8⁺ T cell-mediated elimination. Viral inhibition assays show if the cells are more able to restrict replication in primary CD4⁺ T cells or Tfh, and flow cytometry assays carried out in parallel test if the level of inhibition is explained by Mamu-E or Mamu-1a expression levels on targets. Assessment of the viral reservoir in necropsied animals provides the most direct proof that our combination therpay (group B) is effective in removing latent virus from cells and tissues.

### Statistical Analysis

The primary outcomes of this experiment are measures of control over SIV after ART withdrawal, indicating the superiority of this combination therapy (group B) over therapeutic vaccination alone (group A): (i) weeks to viral rebound after withdrawal of ART, (ii) frequency at which virus fails to return at all, and (iii) the average viral load after withdrawal of ART. Combination therapy tends to provide delayed or absent rebound.

### Example 10. Vaccination Followed by Depletion of Reservoir Cells

In this example SIV-infected and ART-treated macaques are treated with a therapeutic vaccine (cytomegalovirus vectored) followed by reservoir depletion (FIG. 6; groups C, D, and E). Host immune cells elicited by vaccination are able to control the rapidly declining SIV reservoir provided by anti-CD3/CCR5 bsAb; as a result treated, animals manifest lower viral loads after ART removal, as compared to animals that receive vaccine alone.

### Example 11. Heterologous Prime-Boost Vaccination Followed by Depletion of Reservoir Cells

Here several aspects of our combination therapy are improved. Among animals receiving therapeutic vaccination followed by reservoir depletion, the vaccine regimen is improved by administration of a heterologous boost (FIG. 6; group D). The resulting vaccine regimen includes two administrations of RhCMVd10SIVgag (25 and 29 weeks) followed by one administration of Ad26-SlVgag (33 weeks); finally, remaining reservoir cells are targeted with anti-CD3/CCR5 bsAb. In a second group of animals, the same therapeutic regimen is given, but ART therapy is extended by 4 weeks to permit more time for the therapeutic vaccination to take effect (FIG. 6; group E). In a third group of animals, an alternate reservoir-depletion agent is employed (anti-CD4) soon after the beginning of ART therapy and is followed eventually by therapeutic vaccination (FIG. 6; group F).

AdenoSIVgag Vaccine Administration

The two experimental groups receiving a heterologous boost (groups D and E) will receive AdenoSlVgag booster (10¹² vector particles IM) four weeks after a previous RhCMVd10SIVgag administration. The Gag expression cassette consists of sequence-optimized SIVmac239 gag in the expression cassette from the mammalian expression plasmid pcDNA3.1+(Invitrogen, CA, USA). The adenovirus component is created by standard shuttle vector preparation and recombination with an Ad26 backbone in bacterial cells as described. The AdenoSlVgag vector is propagated on C7 or 293 cells and purified by cesium density gradient centrifugation and extensive dialysis.

### Example 12. CCR5 Expression within Macaque and Human T Cell Subsets

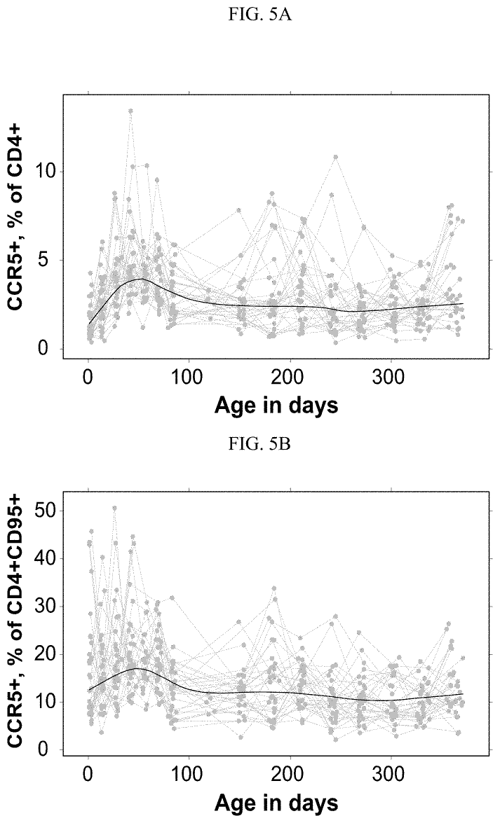

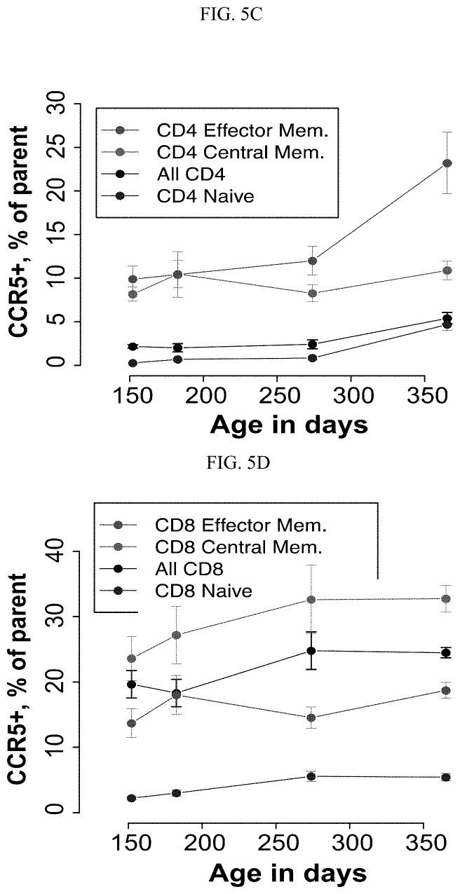

Strategies for elimination of the latent HIV reservoir may range from the very specific (e.g., using novel biomarkers of latently-infected cells) to nonspecific (e.g., myeloablative conditioning). Effective and completely specific strategies are unknown while nonspecific strategies may be associated with side effects due to elimination of uninfected cells. Furthermore, nonspecific strategies may induce homeostatic responses that include cellular activation and proliferation, which could nullify beneficial effects of reservoir reduction. We therefore tracked CCR5 expression on circulating infant macaque T cells throughout the first year of life, in order to determine what fraction of T cells in various subsets would be affected by CCR5 depletion (FIGS. 5A-5D).

We find that CCR5 -expressing cells represent a minority of the infant CD4⁺ T cell population, remaining below 10% of the total in virtually every sample tested (FIG. 5A). Interestingly, the frequency of CCR5⁺ T cells among CD4⁺ cells is lowest at birth, suggesting the possibility that functional cures seen in human infants were abetted by rarity of these cells, which in persistent infections will contribute to the long-lived reservoir. As expected, a greater fraction of memory cells express CCR5 , but this percentage remains below 50% throughout infancy (FIG. 5B). Even among CD28⁻CD95⁺ effector-memory cells, the CD4 subpopulation harboring the greatest number of CCR5⁺ cells, the frequency remains low throughout the first year of life (FIG. 5C). We found that CCR5 expression is more common among infant CD8⁺ T cells, especially CD28⁺CD95⁺ central memory cells. These data suggest that CCR5⁺ cell depletion could have a minor effect on T cell homeostasis, with greatest effects concentrated in CD8⁺ rather than CD4⁺ T cells.

We have shown that depletion of a known HIV reservoir during ART can facilitate “functional cure” of infant macaques treated soon after infection. We then provide examples of how HIV-infected people can be treated at various times after infection using a combination of reservoir-depleting agents and a therapeutic vaccine.

### VI. EXEMPLARY EMBODIMENTS

Exemplary embodiments provided in accordance with the presently disclosed subject matter include, but are not limited to, the claims and the following embodiments:
1. A method for preventing or treating a human immunodeficiency virus (HIV) infection or a simian immunodeficiency virus (SIV) infection in a subject, the method comprising administering to the subject (a) a reservoir-depleting agent that binds to a host protein on a reservoir cell, and (b) one or more antiviral vaccines.
2. The method of embodiment 1, wherein the reservoir cell is a CCR5⁺ cell, a CD4⁺ cell, and/or a CD8⁺ cell.
3. The method of embodiment 1 or 2, wherein the reservoir-depleting agent depletes CCR5⁺ cells.
4. The method of any one of embodiments 1 to 3, wherein the reservoir-depleting agent depletes CD4⁺ cells.
5. The method of any one of embodiments 1 to 4, wherein the reservoir-depleting agent depletes CD8⁺ cells.
6. The method of any one of embodiments 3 to 5, wherein the reservoir-depleting agent depletes CCR5⁺ cells, CD4⁺ cells, and/or CD8⁺ cells in the GI tract, lymph node, spleen, thymus, or lumbar spinal cord.
7. The method of embodiment 6, wherein the reservoir-depleting agent depletes CCR5⁺ cells, CD4⁺ cells, and/or CD8⁺ cells in the GI tract or lymph node.
8. The method of any one of embodiments 1 to 7, wherein the reservoir-depleting agent is an antibody.
9. The method of embodiment 8, wherein the antibody binds to CD4.
10. The method of embodiment 8, wherein the antibody binds to CCR5.
11. The method of any one of embodiments 8 to 10, wherein the antibody is a bispecific antibody.
12. The method of embodiment 11, wherein the bispecific antibody binds to CCR5 and CD3.
13. The method of any one of embodiments 1 to 7, wherein the reservoir-depleting agent is an immunotoxin comprising a CCR5 ligand and a toxin.
14. The method of embodiment 13, wherein the CCR5 ligand is selected from the group consisting of RANTES/CCLS, MIP-lalpha/CCL3, MIP-1beta/CCL4, CCL3L1, and CCL4L1.
15. The method of embodiment 13 or 14, wherein the toxin comprises part or all of a protein selected from the group consisting of diphtheria toxin, Pseudomonas exotoxin, ricin, gelonin, and saponin.
16. The method of any one of embodiments 13 to 15, wherein the toxin is selected from the group consisting of DT385, DT388, DT390, DAB389, DAB486, PE35, PE38, and PE40.
17. The method of any one of embodiments 13 to 16, wherein the toxin is modified to prevent cell entry independent of the CCR5 ligand, to reduce immunogenicity, to improve target-cell toxicity, or to reduce untargeted toxicity.
18. The method of any one of embodiments 1 to 12, wherein the reservoir-depleting agent can simultaneously bind two target host molecules.
19. The method of embodiment 18, wherein the reservoir-depleting agent comprises fused variable domains of immunoglobulin heavy chains and light chains.
20. The method of embodiment 18 or 19, wherein the reservoir-depleting agent is a bispecific T-cell engager, a DART, or a tandem diabody.
21. The method of any one of embodiments 18 to 20, wherein the reservoir-depleting agent binds to CCR5 and CD3.
22. The method of any one of embodiments 1 to 21, wherein the antiviral vaccine is a cytomegalovirus-vectored vaccine, a modified vaccinia ankara B-vectored (MVA-B-vectored) vaccine, a gp120 envelope protein, a gp160 envelope protein, a recombinant adenovirus-5 HIV vaccine, a recombinant adenovirus-26 HIV vaccine, a recombinant adenovirus-35 HIV vaccine, a recombinant simian adenovirus HIV vaccine, a killed whole-HIV-1 vaccine (SAV001), or a canarypox vector.
23. The method of any one of embodiments 1 to 22, wherein the same antiviral vaccine is serially delivered.
24. The method of any one of embodiments 1 to 22, wherein two or more different antiviral vaccines are serially delivered.
25. The method of any one of embodiments 1 to 24, wherein the subject is further administered an antiretroviral therapy (ART).
26. The method of embodiment 25, whereint the ART comprises tenofovir, emtricitabine, and/or dolutegravir.
27. The method of any one of embodiments 1 to 26, wherein the reservoir-depleting agent is administered before the antiviral vaccine.
28. The method of embodiment 27, wherein the reservoir-depleting agent is administered at least one week before the antiviral vaccine.
29. The method of any one of embodiments 1 to 26, wherein the reservoir-depleting agent is administered after the antiviral vaccine.
30. The method of embodiment 29, wherein the reservoir-depleting agent is administered at least one week after the antiviral vaccine.
31. The method of any one of embodiments 1 to 26, wherein the reservoir-depleting agent and the antiviral vaccine are administered substantially at the same time.
32. The method of any one of embodiments 1 to 31, wherein multiple doses of the antiviral vaccine are administered.
33. The method of embodiment 32, wherein the same antiviral vaccine is administered in multiple doses.
34. The method of embodiment 32, wherein two or more different antiviral vaccines are administered in multiple doses.
35. The method of any one of embodiments 1 to 34, wherein the reservoir-depleting agent is administered within 21 days after the subject is exposed to an HIV or a SIV.
36. The method of any one of embodiments 1 to 34, wherein the antiviral vaccine is administered within 21 days after the subject is exposed to an HIV or a SIV.
37. The method of any one of embodiments 25 to 36, wherein the subject is administered the ART before being administered the reservoir-depleting agent or the antiviral vaccine.
38. The method of any one of embodiments 25 to 37, wherein the subject is administered the ART during the entire duration of being administered the reservoir-depleting agent and the antiviral vaccine.
39. The method of any one of embodiments 1 to 38, wherein the subject is a primate.
40. The method of embodiment 39, wherein the subject is a human or a simian.

### VII. REFERENCES

1. UNAIDS. UNAIDS calls on countries to accelerate efforts and close service gaps to end the AIDS epidemic among children and adolescents. 2019 [cited 2019 Dec. 20]; Available from: https://www.unaids.org/en/keywords/children.
2. Haase, A. T., Early events in sexual transmission of HIV and SIV and opportunities for interventions. Annu Rev Med, 2011. 62: p. 127-39.
3. Lifson, J. D., J. L. Rossio, R. Arnaout, L. Li, T. L. Parks, D. K. Schneider, R. F. Kiser, V. J. Coalter, et al., Containment of simian immunodeficiency virus infection: cellular immune responses and protection from rechallenge following transient postinoculation antiretroviral treatment. J Virol, 2000. 74(6): p. 2584-93.
4. Saez-Cirion, A., C. Bacchus, L. Hocqueloux, V. Avettand-Fenoel, I. Girault, C. Lecuroux, V. Potard, P. Versmisse, et al., Post-Treatment HIV-1 Controllers with a Long-Term Virological Remission after the Interruption of Early Initiated Antiretroviral Therapy ANRS VISCONTI Study. PLoS Pathog, 2013. 9(3): p. e1003211.
5. Van Rompay, K. K., L. Durand-Gasselin, L. L. Brignolo, A. S. Ray, K. Abel, T. Cihlar, A. Spinner, C. Jerome, et al., Chronic administration of tenofovir to rhesus macaques from infancy through adulthood and pregnancy: summary of pharmacokinetics and biological and virological effects. Antimicrobial agents and chemotherapy, 2008. 52(9): p. 3144-60.
6. Van Rompay, K. K., M. B. McChesney, N. L. Aguirre, K. A. Schmidt, N. Bischofberger, and M. L. Marthas, Two low doses of tenofovir protect newborn macaques against oral simian immunodeficiency virus infection. J Infect Dis, 2001. 184(4): p. 429-38.
7. Van Rompay, K. K., K. A. Trott, K. Jayashankar, Y. Geng, C. C. LaBranche, J. A. Johnson, G. Landucci, J. Lipscomb, et al., Prolonged tenofovir treatment of macaques infected with K65R reverse transcriptase mutants of SIV results in the development of antiviral immune responses that control virus replication after drug withdrawal. Retrovirology, 2012. 9: p. 57.
8. Hansen, S. G., J. C. Ford, M. S. Lewis, A. B. Ventura, C. M. Hughes, L. Coyne-Johnson, N. Whizin, K. Oswald, et al., Profound early control of highly pathogenic SIV by an effector memory T-cell vaccine. Nature, 2011. 473(7348): p. 523-7.
9. Hansen, S. G., J. B. Sacha, C. M. Hughes, J. C. Ford, B. J. Burwitz, I. Scholz, R. M. Gilbride, M. S. Lewis, et al., Cytomegalovirus vectors violate CD8⁻ T cell epitope recognition paradigms. Science, 2013. 340(6135): p. 1237874.
10. Hansen, S. G., C. Vieville, N. Whizin, L. Coyne-Johnson, D. C. Siess, D. D. Drummond, A. W. Legasse, M. K. Axthelm, et al., Effector memory T cell responses are associated with protection of rhesus monkeys from mucosal simian immunodeficiency virus challenge. Nat Med, 2009. 15(3): p. 293-9.
11. Persaud, D. Functional HIV Cure after Very Early Antiretroviral Therapy in an Infected Infant. in 20th Conference on Retroviruses and Opportunistic Infections. 2013. Atlanta, Ga.: 20th Conference on Retroviruses and Opportunistic Infections.
12. NIH. Diagnosis of HIV Infection in Infants and Children. 2012 [cited 2013 Aug. 29]; Available from: http://aidsinfo.nih.gov/guidelines/html/2/pediatric-arv-guidelines/55/diagnosis-of-hiv-infection-in-infants-and-children.
13. Whitney, J. B., A. L. Hill, S. Sanisetty, P. Penaloza-MacMaster, J. Liu, M. Shetty, L. Parenteau, C. Cabral, et al., Rapid seeding of the viral reservoir prior to SIV viraemia in rhesus monkeys. Nature, 2014. 512(7512): p. 74-7.
14. Kean, W. F., L. Hart, and W. W. Buchanan, Auranofin. Br J Rheumatol, 1997. 36(5): p. 560-72.
15. Hashimoto, K., C. E. Whitehurst, and P. E. Lipsky, Synergistic inhibition of T cell proliferation by gold sodium thiomalate and auranofin. J Rheumatol, 1994. 21(6): p. 1020-6.
16. Han, S., K. Kim, H. Kim, J. Kwon, Y. H. Lee, C. K. Lee, Y. Song, S. J. Lee, and N. Ha, Auranofin inhibits overproduction of pro-inflammatory cytokines, cyclooxygenase expression and PGE2 production in macrophages. Arch Pharm Res, 2008. 31(1): p. 67-74.
17. Kim, T. S., B. Y. Kang, M. H. Lee, Y. K. Choe, and S. Y. Hwang, Inhibition of interleukin-12 production by auranofin, an anti-rheumatic gold compound, deviates CD4(+) T cells from the Th1 to the Th2 pathway. Br J Pharmacol, 2001. 134(3): p. 571-8.
18. Lewis, M. G., S. DaFonseca, N. Chomont, A. T. Palamara, M. Tardugno, A. Mai, M. Collins, W. L. Wagner, et al., Gold drug auranofin restricts the viral reservoir in the monkey AIDS model and induces containment of viral load following ART suspension. Aids, 2011. 25(11): p. 1347-56.
19. Shytaj, I. L., B. Chirullo, W. Wagner, M. G. Ferrari, R. Sgarbanti, A. D. Corte, C. LaBranche, L. Lopalco, et al., Investigational treatment suspension and enhanced cell-mediated immunity at rebound followed by drug-free remission of simian AIDS. Retrovirology, 2013. 10: p. 71.
20. Paiardini, M., B. Cervasi, E. Reyes-Aviles, L. Micci, A.M. Ortiz, A. Chahroudi, C. Vinton, S. N. Gordon, et al., Low levels of SIV infection in sooty mangabey central memory CD(4)(+) T cells are associated with limited CCR5 expression. Nat Med, 2011. 17(7): p. 830-6.
21. Pandrea, I., C. Apetrei, S. Gordon, J. Barbercheck, J. Dufour, R. Bohm, B. Sumpter, P. Rogues, et al., Paucity of CD4⁺CCR5⁺ T cells is a typical feature of natural SIV hosts. Blood, 2007. 109(3): p. 1069-76.
22. Taaffe, J. E., S. E. Bosinger, G. Q. Del Prete, J. G. Else, S. Ratcliffe, C. D. Ward, T. Migone, M. Paiardini, and G. Silvestri, CCR5 blockade is well tolerated and induces changes in the tissue distribution of CCR5⁺ and CD25⁺ T cells in healthy, SIV-uninfected rhesus macaques. J Med Primatol, 2012. 41(1): p. 24-42.
23. Eisele, E. and R. F. Siliciano, Redefining the viral reservoirs that prevent HIV-1 eradication. Immunity, 2012. 37(3): p. 377-88.
24. Del Prete, G., R. Geleziunas, J. Hesselgesser, B. Li, J. Hattersley, B. Lu, J. Zheng, J. Smedley, et al., Comparison of Combination ART Regimens for Viral Suppression in SIV-Infected Rhesus Macaques, in Conference on Retroviruses and Opportunistic Infections. 2014: Boston, Mass. p. 295.
25. Del Prete, G. Q. and J. D. Lifson, Considerations in the development of nonhuman primate models of combination antiretroviral therapy for studies of AIDS virus suppression, residual virus, and curative strategies. Curr Opin HIV AIDS, 2013. 8(4): p. 262-72.
26. Merriam, D., C. Chen, G. Mendez-Lagares, K. A. Rogers, A. J. Michaels, J. Yan, P. Casaz, K. A. Reimann, et al., Depletion of Gut-Resident CCR5(+) Cells for HIV Cure Strategies. AIDS Res Hum Retroviruses, 2017. 33(S1): p. S70-S80.
27. Okoye, A. A., S. G. Hansen, M. Vaidya, Y. Fukazawa, H. Park, D. M. Duell, R. Lum, C. M. Hughes, et al., Early antiretroviral therapy limits SIV reservoir establishment to delay or prevent post-treatment viral rebound. Nat Med, 2018. 24(9): p. 1430-1440.
28. Labrijn, A. F., J. I. Meesters, B. E. de Goeij, E. T. van den Bremer, J. Neijssen, M. D. van Kampen, K. Strumane, S. Verploegen, et al., Efficient generation of stable bispecific IgG1 by controlled Fab-arm exchange. Proc Natl Acad Sci USA, 2013. 110(13): p. 5145-50.
29. Michaelsson, J., J. E. Mold, J. M. McCune, and D. F. Nixon, Regulation of T cell responses in the developing human fetus. J Immunol, 2006. 176(10): p. 5741-8.
30. Nilsson, J., A. Boasso, P. A. Velilla, R. Zhang, M. Vaccari, G. Franchini, G. M. Shearer, J. Andersson, and C. Chougnet, HIV-1-driven regulatory T-cell accumulation in lymphoid tissues is associated with disease progression in HIV/AIDS. Blood, 2006. 108(12): p. 3808-17.
31. Nilsson, J., S. Kinloch-de-Loes, A. Granath, A. Sonnerborg, L. E. Goh, and J. Andersson, Early immune activation in gut-associated and peripheral lymphoid tissue during acute HIV infection. Aids, 2007. 21(5): p. 565-74.
32. Hong, J. J., M. R. Reynolds, T. L. Mattila, A. Hage, D. I. Watkins, C. J. Miller, and P. J. Skinner, Localized populations of CD8 MHC class I tetramer SIV-specific T cells in lymphoid follicles and genital epithelium. PLoS One, 2009. 4(1): p. e4131.
33. Hancock, G., H. Yang, E. Yorke, E. Wainwright, V. Bourne, A. Frisbee, T. L. Payne, M. Berrong, et al., Identification of effective subdominant anti-HIV-1 CD8⁺ T cells within entire post-infection and post-vaccination immune responses. PLoS Pathog, 2015. 11(2): p. e1004658.
34. Slichter, C. K., D. P. Friedrich, R. J. Smith, P. N. Walsh, G. Mize, J. L. Czartoski, M. J. McElrath, and N. Frahm, Measuring inhibition of HIV replication by ex vivo CD8(+) T cells. J Immunol Methods, 2014. 404: p. 71-80.
35. North, T. W., J. Higgins, J. D. Deere, T. L. Hayes, A. Villalobos, L. Adamson, B. L. Shacklett, R. F. Schinazi, and P. A. Luciw, Viral sanctuaries during highly active antiretroviral therapy in a nonhuman primate model for AIDS. J Virol, 2010. 84(6): p. 2913-22.
36. Hartigan-O'Connor, D., A. Amalfitano, and J. S. Chamberlain, Improved production of gutted adenovirus in cells expressing adenovirus preterminal protein and DNA polymerase. J Virol, 1999. 73(9): p. 7835-41.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence reference numbers cited herein are hereby incorporated by reference in their entirety for all purposes.

1. A method for preventing or treating a human immunodeficiency virus (HIV) infection or a simian immunodeficiency virus (SIV) infection in a subject, the method comprising administering to the subject (a) a reservoir-depleting agent that binds to a host protein on a reservoir cell, and (b) one or more antiviral vaccines.

2. The method of claim 1, wherein the reservoir cell is a CCR5+ cell, a CD4+ cell, and/or a CD8+ cell.

3. The method of claim 1, wherein the reservoir-depleting agent depletes CCR5+ cells.

4. The method of claim 1, wherein the reservoir-depleting agent depletes CD4+ cells.

5. The method of claim 1, wherein the reservoir-depleting agent depletes CD8+ cells.

6. The method of claim 1, wherein the reservoir-depleting agent depletes CCR5+ cells, CD4+ cells, and/or CD8+ cells in the GI tract, lymph node, spleen, thymus, or lumbar spinal cord.

7. (canceled)

8. The method of claim 1, wherein the reservoir-depleting agent is an antibody.

9. The method of claim 8, wherein the antibody binds to CD4.

10. The method of claim 8, wherein the antibody binds to CCR5.

11. The method of claim 8, wherein the antibody is a bispecific antibody.

12. The method of claim 11, wherein the bispecific antibody binds to CCR5 and CD3.

13. The method of claim 1, wherein the reservoir-depleting agent is an immunotoxin comprising a CCR5 ligand and a toxin.

14. The method of claim 13, wherein the CCR5 ligand is selected from the group consisting of RANTE5/CCL5, MIP-1alpha/CCL3, MIP-1beta/CCL4, CCL3L1, and CC4L1.

15. The method of claim 13, wherein the toxin comprises part or all of a protein selected from the group consisting of diphtheria toxin, Pseudomonas exotoxin, ricin, gelonin, and saponin.

16. The method of claim 13, wherein the toxin is selected from the group consisting of DT385, DT388, DT390, DAB389, DAB486, PE35, PE38, and PE40.

17. (canceled)

18. The method of claim 1, wherein the reservoir-depleting agent can simultaneously bind two target host molecules.

19. (canceled)

20. The method of claim 18, wherein the reservoir-depleting agent is a bispecific T-cell engager, a DART, or a tandem diabody.

21. The method of claim 18, wherein the reservoir-depleting agent binds to CCR5 and CD3.

22. The method of claim 1, wherein the antiviral vaccine is a cytomegalovirus-vectored vaccine, a modified vaccinia ankara B-vectored (MVA-B-vectored) vaccine, a gp120 envelope protein, a gp160 envelope protein, a recombinant adenovirus-5 HIV vaccine, a recombinant adenovirus-26 HIV vaccine, a recombinant adenovirus-35 HIV vaccine, a recombinant simian adenovirus HIV vaccine, a killed whole-HIV-1 vaccine (SAV001), or a canarypox vector.

23. The method of claim 1, wherein the same antiviral vaccine is serially delivered.

24. The method of claim 1, wherein two or more different antiviral vaccines are serially delivered.

25. The method of claim 1, wherein the subject is further administered an antiretroviral therapy (ART).

26. (canceled)

27. The method of claim 1, wherein the reservoir-depleting agent is administered before the antiviral vaccine.

28. The method of claim 27, wherein the reservoir-depleting agent is administered at least one week before the antiviral vaccine.

29. The method of claim 1, wherein the reservoir-depleting agent is administered after the antiviral vaccine.

30. The method of claim 29, wherein the reservoir-depleting agent is administered at least one week after the antiviral vaccine.

31. The method of claim 1, wherein the reservoir-depleting agent and the antiviral vaccine are administered substantially at the same time.

32-34. (canceled)

35. The method of claim 1, wherein the reservoir-depleting agent is administered within 21 days after the subject is exposed to an HIV or a SIV.

36. The method of claim 1, wherein the antiviral vaccine is administered within 21 days after the subject is exposed to an HIV or a SIV.

37-40. (canceled)
